(19) United States
(12) Patent Application Publication
(10) Pub. No.: US 2025/0316377 A1
(43) Pub. Date: Oct. 9, 2025

(54) PREDICTING ALBUMINURIA USING MACHINE LEARNING

(71) Applicant: ASTRAZENECA AB, Södertälje (SE)

(72) Inventors: Nils SVANGÅRD, Södertälje (SE); Peter GREASLEY, Södertälje (SE); Philip AMBERY, Södertälje (SE); Shameer KHADER, Wilmington, DE (US)

(21) Appl. No.: 18/855,182

(22) PCT Filed: May 12, 2023
(86) PCT No.: PCT/IB2023/000267

§ 371 Date: Oct. 8, 2024

Related U.S. Application Data
(60) Provisional application No. 63/343,778, filed on May 19, 2022.
(60) Provisional application No. 63/456,855, filed on Apr. 4, 2023.

(51) Int. Cl. G16H 50/20; G16H 10/60

(52) U.S. Cl. CPC G16H 50/20; G16H 10/60

(57) ABSTRACT

An example embodiment may involve obtaining, by a computing system, an observation of demographic values of an individual, vital sign values of the individual, and blood test values of the individual: applying, by the computing system, a machine learning model to the observation, wherein the machine learning model was trained with a training data set, wherein the training data set contained observations of corresponding demographic values, vital sign values, blood test values, and either urine albumin-to-creatinine ratio (UACR) values or urine protein-to-creatinine ratio (UPR) values for a plurality of individuals, and wherein the machine learning model is configured to provide predictions of whether further observations are indicative of undiagnosed albuminuria or proteinuria; and providing, by the computing system, a prediction of whether the individual exhibits undiagnosed albuminuria or proteinuria based on the observation.

### CROSS-REFERENCE TO RELATED APPLICATIONS

This application claims priority to U.S. provisional patent application No. 63/456,855, filed Apr. 4, 2023 and U.S. provisional patent application No. 63/343,778, filed May 19, 2022, both of which are hereby incorporated by reference in their entirety.

### BACKGROUND

Albumin is a protein secreted by the liver and found in the blood. A properly-functioning kidney does not allow albumin to pass from the blood into the urine. Albuminuria is a pathological condition wherein albumin is abnormally present in the urine, and is an indicator of chronic kidney disease (CKD). Left untreated, CKD can lead to kidney failure and death. The measurement of albumin in the urine can be used to assess the progression of CKD and serves as an indication that a patient is a candidate for treatment and is also a surrogate marker for treatment effect. While blood tests are commonly used to detect the presence of other markers of CKD, relatively few patients are screened for albuminuria. Particularly, albuminuria testing requires collection of a urine sample, which is not commonly ordered for patients without diabetes or other kidney-related conditions. Consequently, some patients can suffer from albuminuria for a long period of time (e.g., months or years) without receiving the proper diagnosis or necessary treatment.

### SUMMARY

Urine albumin levels can be determined from samples using a urine albumin-to-creatinine ratio (UACR) test. A result in the range of 30-300 mg/g is referred to as microalbuminuria, and a result greater than 300 mg/g is referred to as macroalbuminuria. Nonetheless, this cutoff of 300 mg/g is somewhat arbitrary and not indicative of a clinically significant inflection point. In some clinical scenarios, the cutoff may vary, with values anywhere between 150 mg/g and 700 mg/g.

Early detection of either category of albuminuria and subsequent medical intervention can lower the risk of developing worsening kidney function, including CKD progression and progression to end-stage renal disease (ESRD). Early detection can also lead to reduced risk of incident cardiovascular events even when albuminuria is detected in patients with normal renal function. Given that UACR results are not available for many patients, it is beneficial to improve albuminuria screening by identifying patients with suspected undiagnosed albuminuria based on more widely-available biomarkers. Data for these biomarkers already exist in patient's electronic health records (e.g., from demographic data, health data, blood tests, etc.). Therefore, it is possible to predict which patients are likely to have undiagnosed albuminuria based on these records and/or other available medical information. Once these patients are identified, they can be contacted proactively for UACR testing and treatment, should those tests indicate albuminuria.

Consequently, application of the embodiments herein can identify albuminuria earlier than it would have otherwise been detected. Early detection and the subsequent early medical intervention can reduce the impact of CKD and prevent eventual kidney failure. Further, identified patients may also be recruited for medical studies or clinical trials of new treatments and/or pharmaceuticals that may be able to provide improved treatment options and health outcomes.

To address these issues, these embodiments employ a machine learning model that is trained on a combination of patient demographics, vital signs, blood tests, and/or other medical information. The training results in a classifier that predicts UACR levels. In some cases, the machine learning model is based on gradient boosting technology, but other underlying technologies (e.g., artificial neural networks or expert systems) may be used instead or in conjunction with gradient boosting. Such a model has been applied to clinical data and is shown to be effective at predicting UACR levels for a wide range of patients. In some embodiments, urine protein-to-creatinine ratio (UPR) levels can be used in place of or to derive UACR values. These UACR and UPR levels may be calculated from separate urine albumin, urine creatinine, and/or urine protein measurements.

Accordingly, a first example embodiment involves obtaining, by a computing system, a training data set, wherein the training data set contains observations of corresponding demographic values, vital sign values, blood test values, and either UACR values or UPR values for a plurality of individuals; and applying, by the computing system, a machine learning trainer to the training data set, wherein the machine learning trainer produces a machine learning model, and wherein the machine learning model is configured to take a new observation of new demographic values, new vital sign values, and new blood test values as input and provide a prediction of whether an individual exhibiting the new observation has undiagnosed albuminuria or proteinuria.

A second example embodiment involves obtaining, by a computing system, an observation of demographic values of an individual, vital sign values of the individual, and blood test values of the individual; applying, by the computing system, a machine learning model to the observation, wherein the machine learning model was trained with a training data set, wherein the training data set contained observations of corresponding demographic values, vital sign values, blood test values, and either UACR values or UPR values for a plurality of individuals, and wherein the machine learning model is configured to provide predictions of whether further observations are indicative of undiagnosed albuminuria or proteinuria; and providing, by the computing system, a prediction of whether the individual exhibits undiagnosed albuminuria or proteinuria based on the observation.

A third example embodiment involves obtaining, by a computing system, a training data set, wherein the training data set contains observations of corresponding demographic values, vital sign values, blood test values, and either UACR values or UPR values for a plurality of individuals; and applying, by the computing system, a quantile regression machine learning trainer to the training data set, wherein the quantile regression machine learning trainer produces a quantile regression machine learning model, and wherein the quantile regression machine learning model is configured to take a quantile and a new observation of new demographic values, new vital sign values, and new blood test values as input and provide a prediction of a UACR or UPR value at the quantile for an individual exhibiting the new observation.

A fourth example embodiment involves obtaining, by a computing system, a quantile and an observation of demographic values of an individual, vital sign values of the individual, and blood test values of the individual; applying, by the computing system, a quantile regression machine learning model to the observation, wherein the quantile regression machine learning model was trained with a training data set, wherein the training data set contained observations of corresponding demographic values, vital sign values, blood test values, and either UACR values or UPR values for a plurality of individuals, and wherein the quantile regression machine learning model is configured to provide predictions of UACR or UPR values at one or more quantiles for further observations; based on the observation and for the individual, providing, by the computing system, a prediction of a UACR or UPR value at the quantile.

In a fifth example embodiment, an article of manufacture includes a non-transitory computer-readable medium, having stored thereon program instructions that, upon execution by a computing system, cause the computing system to perform operations in accordance with the first, second, third, and/or fourth example embodiment.

In a sixth example embodiment, a computing system includes at least one processor, as well as memory and program instructions. The program instructions may be stored in the memory, and upon execution by the at least one processor, cause the computing system to perform operations in accordance with the first, second, third, and/or fourth example embodiment.

In a seventh example embodiment, a system includes various means for carrying out each of the operations of the first, second, third, and/or fourth example embodiment.

These, as well as other embodiments, aspects, advantages, and alternatives, will become apparent to those of ordinary skill in the art by reading the following detailed description, with reference where appropriate to the accompanying drawings. Further, this summary and other descriptions and figures provided herein are intended to illustrate embodiments by way of example only and, as such, that numerous variations are possible. For instance, structural elements and process steps can be rearranged, combined, distributed, eliminated, or otherwise changed, while remaining within the scope of the embodiments as claimed.

### BRIEF DESCRIPTION OF THE DRAWINGS

FIG. 1 illustrates a schematic drawing of a computing device, in accordance with example embodiments.

FIG. 2 illustrates a schematic drawing of a server device cluster, in accordance with example embodiments.

FIG. 3 illustrates the training and use of a gradient boosting model, in accordance with example embodiments.

FIG. 4 depicts interactions between two biomarkers for CKD, in accordance with example embodiments.

FIG. 5 depicts use of a machine learning model for predicting albuminuria, in accordance with example embodiments.

FIG. 6 depicts candidate features for training a machine learning model, in accordance with example embodiments.

FIG. 7 depicts an overview of two training data sets, in accordance with example embodiments.

FIG. 8 depicts plots of UACR distributions for the two training data sets, in accordance with example embodiments.

FIG. 9 depicts ROC and precision-recall curves for a trained macroalbuminuria model, in accordance with example embodiments.

FIG. 10 depicts ROC and precision-recall curves for a trained microalbuminuria model, in accordance with example embodiments.

FIGS. 11A and 11B depict Shapley values for a trained machine learning model, in accordance with example embodiments.

FIGS. 12A, 12B, 12C, and 12D depicts results of model evaluation, in accordance with example embodiments.

FIGS. 13 and 14 are flow charts, in accordance with example embodiments.

FIG. 15 depicts use of a quantile regression machine learning model for predicting UACR quantiles, in accordance with example embodiments.

FIG. 16 depicts plots of predicted survivability for UACR medians and true UACR values, in accordance with example embodiments.

FIGS. 17 and 18 are flow charts, in accordance with example embodiments.

FIGS. 19A and 19B depict a user interface and workflow, in accordance with example embodiments.

### DETAILED DESCRIPTION

Example methods, devices, and systems are described herein. It should be understood that the words “example” and “exemplary” are used herein to mean “serving as an example, instance, or illustration.” Any embodiment or feature described herein as being an “example” or “exemplary” is not necessarily to be construed as preferred or advantageous over other embodiments or features unless stated as such. Thus, other embodiments can be utilized and other changes can be made without departing from the scope of the subject matter presented herein.

Accordingly, the example embodiments described herein are not meant to be limiting. It will be readily understood that the aspects of the present disclosure, as generally described herein, and illustrated in the figures, can be arranged, substituted, combined, separated, and designed in a wide variety of different configurations. For example, the separation of features into “client” and “server” components may occur in a number of ways.

Further, unless context suggests otherwise, the features illustrated in each of the figures may be used in combination with one another. Thus, the figures should be generally viewed as component aspects of one or more overall embodiments, with the understanding that not all illustrated features are necessary for each embodiment.

Additionally, any enumeration of elements, blocks, or steps in this specification or the claims is for purposes of clarity. Thus, such enumeration should not be interpreted to require or imply that these elements, blocks, or steps adhere to a particular arrangement or are carried out in a particular order.

### I. Example Computing Devices and Cloud-Based Computing Environments

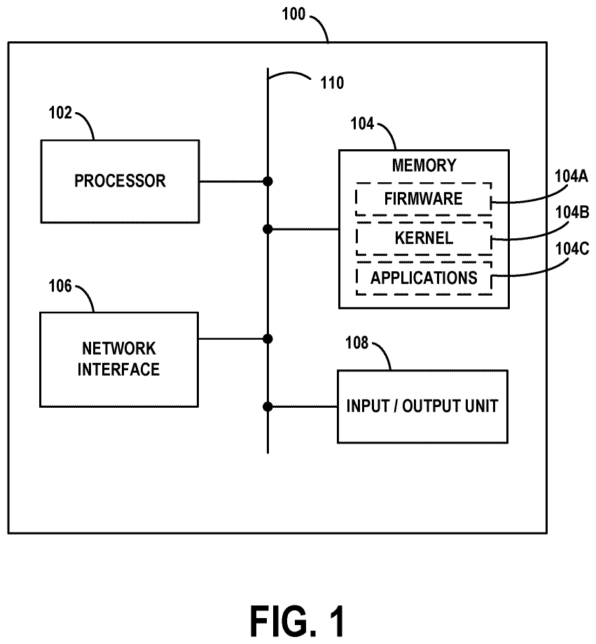

FIG. 1 is a simplified block diagram exemplifying a computing device 100, illustrating some of the components that could be included in a computing device arranged to operate in accordance with the embodiments herein. Computing device 100 could be a client device (e.g., a device actively operated by a user), a server device (e.g., a device that provides computational services to client devices), or some other type of computational platform. Some server devices may operate as client devices from time to time in order to perform particular operations, and some client devices may incorporate server features.

In this example, computing device 100 includes processor 102, memory 104, network interface 106, and input/output unit 108, all of which may be coupled by system bus 110 or a similar mechanism. In some embodiments, computing device 100 may include other components and/or peripheral devices (e.g., detachable storage, printers, and so on).

Processor 102 may be one or more of any type of computer processing element, such as a central processing unit (CPU), a co-processor (e.g., a mathematics, graphics, or encryption co-processor), a digital signal processor (DSP), a network processor, and/or a form of integrated circuit or controller that performs processor operations. In some cases, processor 102 may be one or more single-core processors. In other cases, processor 102 may be one or more multi-core processors with multiple independent processing units. Processor 102 may also include register memory for temporarily storing instructions being executed and related data, as well as cache memory for temporarily storing recently-used instructions and data.

Memory 104 may be any form of computer-usable memory, including but not limited to random access memory (RAM), read-only memory (ROM), and non-volatile memory (e.g., flash memory, hard disk drives, solid state drives, and/or tape storage). Thus, memory 104 represents both main memory units, as well as long-term storage. Other types of memory may include biological memory.

Memory 104 may store program instructions and/or data on which program instructions may operate. By way of example, memory 104 may store these program instructions on a non-transitory, computer-readable medium, such that the instructions are executable by processor 102 to carry out any of the methods, processes, or operations disclosed in this specification or the accompanying drawings.

As shown in FIG. 1, memory 104 may include firmware 104A, kernel 104B, and/or applications 104C. Firmware 104A may be program code used to boot or otherwise initiate some or all of computing device 100. Kernel 104B may be an operating system, including modules for memory management, scheduling, and management of processes, input/output, and communication. Kernel 104B may also include device drivers that allow the operating system to communicate with the hardware modules (e.g., memory units, networking interfaces, ports, and buses) of computing device 100. Applications 104C may be one or more user-space software programs, such as web browsers or email clients, as well as any software libraries used by these programs. Memory 104 may also store data used by these and other programs and applications.

Network interface 106 may take the form of one or more wireline interfaces, such as Ethernet (e.g., Fast Ethernet, Gigabit Ethernet, and so on). Network interface 106 may also support communication over one or more non-Ethernet media, such as coaxial cables or power lines, or over wide-area media, such as Synchronous Optical Networking (SONET) or software-define wide-area networking (SD-WAN) technologies. Network interface 106 may additionally take the form of one or more wireless interfaces, such as IEEE 802.11 (Wifi), BLUETOOTH®, global positioning system (GPS), or a wide-area wireless interface. However, other forms of physical layer interfaces and other types of standard or proprietary communication protocols may be used over network interface 106. Furthermore, network interface 106 may comprise multiple physical interfaces. For instance, some embodiments of computing device 100 may include Ethernet, BLUETOOTH®, and Wifi interfaces.

Input/output unit 108 may facilitate user and peripheral device interaction with computing device 100. Input/output unit 108 may include one or more types of input devices, such as a keyboard, a mouse, a touch screen, and so on. Similarly, input/output unit 108 may include one or more types of output devices, such as a screen, monitor, printer, and/or one or more light emitting diodes (LEDs). Additionally or alternatively, computing device 100 may communicate with other devices using a universal serial bus (USB) or high-definition multimedia interface (HDMI) port interface, for example.

One or more computing devices like computing device 100 may be deployed to support the embodiments herein. The exact physical location, connectivity, and configuration of these computing devices may be unknown and/or unimportant to client devices. Accordingly, the computing devices may be referred to as “cloud-based” devices that may be housed at various remote data center locations.

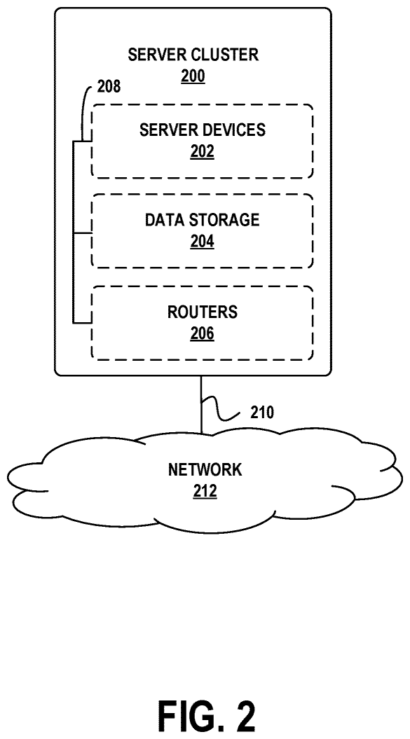

FIG. 2 depicts a cloud-based server cluster 200 in accordance with example embodiments. In FIG. 2, operations of a computing device (e.g., computing device 100) may be distributed between server devices 202, data storage 204, and routers 206, all of which may be connected by local cluster network 208. The number of server devices 202, data storages 204, and routers 206 in server cluster 200 may depend on the computing task(s) and/or applications assigned to server cluster 200.

For example, server devices 202 can be configured to perform various computing tasks of computing device 100. Thus, computing tasks can be distributed among one or more of server devices 202. To the extent that these computing tasks can be performed in parallel, such a distribution of tasks may reduce the total time to complete these tasks and return a result. For purposes of simplicity, both server cluster 200 and individual server devices 202 may be referred to as a “server device.” This nomenclature should be understood to imply that one or more distinct server devices, data storage devices, and cluster routers may be involved in server device operations.

Data storage 204 may be data storage arrays that include drive array controllers configured to manage read and write access to groups of hard disk drives and/or solid state drives. The drive array controllers, alone or in conjunction with server devices 202, may also be configured to manage backup or redundant copies of the data stored in data storage 204 to protect against drive failures or other types of failures that prevent one or more of server devices 202 from accessing units of data storage 204. Other types of memory aside from drives may be used.

Routers 206 may include networking equipment configured to provide internal and external communications for server cluster 200. For example, routers 206 may include one or more packet-switching and/or routing devices (including switches and/or gateways) configured to provide (i) network communications between server devices 202 and data storage 204 via local cluster network 208, and/or (ii) network communications between server cluster 200 and other devices via communication link 210 to network 212.

Additionally, the configuration of routers 206 can be based at least in part on the data communication requirements of server devices 202 and data storage 204, the latency and throughput of the local cluster network 208, the latency, throughput, and cost of communication link 210, and/or other factors that may contribute to the cost, speed, fault-tolerance, resiliency, efficiency, and/or other design goals of the system architecture.

As a possible example, data storage 204 may include any form of database, such as a structured query language (SQL) database. Various types of data structures may store the information in such a database, including but not limited to tables, arrays, lists, trees, and tuples. Furthermore, any databases in data storage 204 may be monolithic or distributed across multiple physical devices.

Server devices 202 may be configured to transmit data to and receive data from data storage 204. This transmission and retrieval may take the form of SQL queries or other types of database queries, and the output of such queries, respectively. Additional text, images, video, and/or audio may be included as well. Furthermore, server devices 202 may organize the received data into web page or web application representations, or for use by a software application in some other fashion. Such a representation may take the form of a markup language, such as HTML, the extensible Markup Language (XML), or some other standardized or proprietary format.

Moreover, server devices 202 may have the capability of executing various types of computerized scripting languages, such as but not limited to Perl, Python, PHP Hypertext Preprocessor (PHP), Active Server Pages (ASP), JAVASCRIPT®, and so on. Computer program code written in these languages may facilitate the providing of web pages to client devices, as well as client device interaction with the web pages. Alternatively or additionally, JAVA® may be used to facilitate generation of web pages and/or to provide web application functionality.

### II. Example Gradient Boosting Models

Gradient boosting algorithms are machine learning techniques that can be used to develop prediction models for multi-dimensional data sets. For convenience, these sets are often represented in matrix form using columns and rows. One or more columns represent input variables, and a further column represents an output variable. The output variable is an unknown function of one or more of the input variables. The rows represent observations of input variables and their corresponding output variables, usually based on real-world data. In many cases, the number of rows can be quite large, in the hundreds, thousands, or more. The machine learning process involves training the gradient boosting model to be able to predict the output variable for new observations of the input variables. In other words, the model attempts to learn or at least approximate the unknown function from the existing instances of input variables and their corresponding output variables.

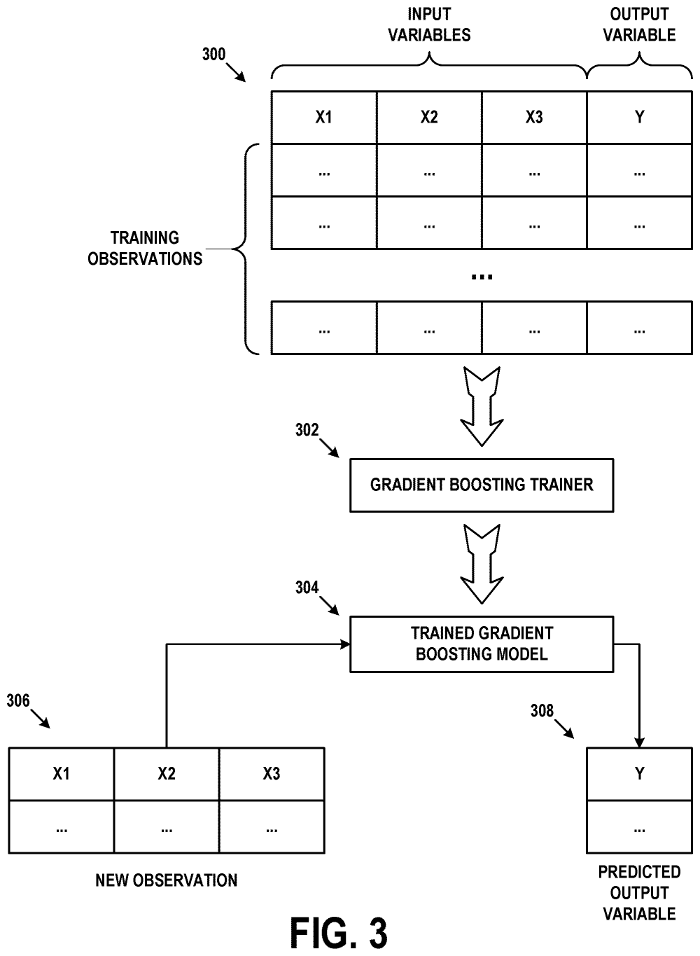

FIG. 3 further illustrates these concepts. Training data set 300 includes a set of training observations (rows), each consisting of input variables X1, X2, and X3 and their corresponding output variable, Y. These input and output variables are related by way of some unknown function ƒ, where Y=ƒ(X1, X2, X3). The output variable Y can take various forms, such as integer or real numbers, text, or Boolean values.

Training data set 300 may be gathered from actual patient medical data, e.g., from health professionals, hospitals, clinical trials or other sources. In such a training data set, the values of the output variable for each observation is expected to be known, but not every value of the input variables needs to be present—e.g., the training data set may be sparsely populated.

Training data set 300 is provided to gradient boosting trainer 302, which applies one or more training techniques to produce gradient boosting model 304. Gradient boosting model 304 may be an algorithm, or set of parameters to control the behavior of an algorithm, that can be used to apply an approximation of unknown function ƒ to new observations of the input variables.

Thus, gradient boosting model 304 may receive new observation 306 and produce predicted output variable 308. The accuracy of such predictions can vary based on the operation of gradient boosting trainer 302 and the quality of training data set 300. The goal is for gradient boosting model 304 to be as accurate as reasonably possible given a sufficiently rich training data set and a reasonable amount of time to spend on the training. This accuracy may be measured in various ways, as described in more detail below.

The operation of gradient boosting trainer 302 may involve training a set of decision trees (colloquially referred to as a “forest”), each of a limited depth or with a limited number of leaves. Thus, these trees are weak learners in that they generally do not take into consideration all available information in the training data set, and therefore their individual predictions may or may not have a high degree of accuracy. But gradient boosting makes overall predictions based on a weighting of the predictions from the individual trees. These overall predictions take into account most if not all of the training data set and therefore are likely to be more accurate than predictions from any of the individual trees.

But unlike a random forest, in which each tree is independent of the others, the construction of subsequent trees in a gradient boosting model can be based on the errors (or residuals) of one or more of the previously-constructed trees. In some cases, subsequent trees that compensate well for the errors of previous trees are given more weight toward the overall predictions, while in others all trees may be equally-weighted. Gradient boosting continues to construct trees in this fashion until it constructs a pre-determined number of trees or the new trees fail to improve the accuracy of the predictions by more than a pre-determined margin.

When the output variable takes on a continuous value, e.g., an integer falling with some range, trees are constructed based on the magnitude of residuals between actual values of the training data output variables and the associated predicted values. This may be referred to as gradient boosting for regression. In some cases, these residuals are called “pseudo-residuals” in order to differentiate gradient boosting from linear regression, but terms “residuals” and “pseudo-residuals” will be used interchangeably herein. The initial predictions for each observation i, p_{i,0}, may take on the same value p₀, such as the average of some or all output variables in the training data set. In other words, p₀=p_{i,0}∀ᵢ.

Here, the notation p_{i,j} refers to the prediction for observation i made by using trees 0 through n (see below for more details on how predictions are calculated using multiple trees). The initial prediction, p₀, may take the form of a single node rather than a tree, since it is commonly based only on values of the output variable.

In any event, the trees are constructed to predict the values of the residuals. The non-leaf nodes of the trees represent conditions of the input variables. For example, the root node in a tree constructed from training data set 300 might represent the condition X2>5 such that when this condition is true the node's left branch is followed, and when this condition is false the node's right branch is followed. Either of these branches might lead to another non-leaf node representing a condition or a leaf node representing a residual. More than two branches may be present, but binary trees are used in the examples herein for sake of convenience.

Tree construction may be based on various algorithms used for decision trees. In some cases, this may involve selecting an input variable and possibly an associated cutoff value that is based on entropy or Gini impurity. The cutoff value is selected so that it divides the values of the input variable in a fashion that makes the input variable reasonably predictive of the output variable. Then, the input variables are arranged as nodes in the tree with more predictive input variables generally being placed higher in the tree (e.g., closer to the root node). In some cases, randomness may be added to the process of determining where to place the input variables in the tree.

Since the number of observations is usually much greater than the number of leaves in a limited-size tree, the residuals of each observation that leads to the same leaf are typically averaged then placed in the leaf. Thus, a leaf can represent an aggregate residual, r_{i,0}, for a number of observations. Here, the notation r_{i,j} refers to the residual for observation i made in from using trees 0 through n (see below for more detail on how residuals are calculated using multiple trees).

Iterative predictions are then made for the observations in the training data set. Each prediction of the first iteration, p_{i,1}, involves traversing the tree 1 for an observation until reaching a leaf, and then adding that leaf's residual, r_{i,0}, to the initial prediction in accordance with a learning rate 0<α<1. In other words, p_{i,1}=p_{i,0}+αr_{i,0}. The learning rate helps prevent overfitting the training data set and allows small steps to be taken toward a higher prediction accuracy.

From the predictions p_{i,1}, new residuals r_{i,1} are calculated. Again, the residuals are based on differences between the actual output variable values in the training data set and the associated predicted values, with possible aggregation as described above. It is expected that these new residuals will generally be smaller than those of r_{i,0}, but this is not always the case for every residual.

The next tree, tree 2, may be constructed based on these new residuals. This tree may have the same structure as tree 1 or may be structured differently with nodes representing the input variables appearing in different locations (e.g., using randomness).

Then, each prediction of the second iteration, p_{i,2}, involves traversing both tree 1 and tree 2 for an observation until reaching their respective leaves, and then adding the associated residuals, r_{i,0} and r_{i,1}, to the initial prediction in accordance with the learning rate. In other words, p_{i,2}=p_{i,0}+αr_{i,0}+αr_{i,1}.

From the predictions p_{i,2}, new residuals r_{i,2} are calculated. It is expected that these residuals will continue getting smaller as the number of trees grows.

The process of constructing new trees based on residuals and making new predictions continues until, as noted above, a pre-determined number of trees are constructed or adding new trees fails to reduce the size of the residuals by more than a pre-determined margin. At this point, the training ends and the trained gradient boosting model is ready to make predictions for new observations of input variables.

Given such a new observation, the model begins with the initial prediction, p₀, and traverses all of the trees in accordance with the values of the input variables, adding the resulting residuals. Thus, assuming n trees in addition to the initial node, the predicted value of the output variable for a new observation is

$${p}_{new} = {p}_{0} + α {\sum }_{j = 0}^{n} {r}_{j} ,$$

where r₀ is the initial residual and rⱼ is for 1≤j≤n is the residual the jth tree for this observation.

Gradient boosting can also be used to predict the value of the output variable from a limited number of possible values. For example, when the output variable is Boolean, gradient boosting can be used to train a binary classifier. This may be referred to as gradient boosting for classification.

In this case, the predictions can be based on, across all observations in the training data set, (i) the natural log of the odds that the output variable is true, and (ii) the probability that the output variable is true. For instance, suppose that there are 100 observations with 70 being “true” and 30 being “false”. The natural logarithm of the odds that an observation is true would be ln(70/30)=0.847, while the probability that the output variable is true is 0.7.

The natural logarithm of the odds (0.847) is used as the initial prediction for all observations, and the probability (0.7) is used to calculate the residuals. Since 0.7 is greater than 0.5, the initial predictions are “true” for all observations (note that values other than 0.5 can be used as a cutoff in this process). Clearly, these initial predictions are not accurate, as indicated by their residuals. Assigning a value of 1.0 for true and 0.0 for false, the residuals will be 0.3 for each observation with an output variable that is “true” and −0.7 for each observation with an output variable that is “false”.

Further, the residuals are typically transformed to generate the output values of the leaves. This is because they are in terms of a probability while the predictions are in terms of the natural logarithm of the odds. An example transformation for a leaf with residuals rₖ as corresponding predicted probabilities ρₖ is:

$$\frac{{\sum }_{k} {r}_{k}}{{\sum }_{k} ( {ρ}_{k} ) ( 1 - {ρ}_{k} )}$$

Then these output values for the leaves are scaled by the learning rate and added to the initial predictions. Since these predictions are still in the form of the natural log of the odds, then can be converted to the probability form used by the residuals through application the logistic function. For instance, a predicted value of p would have a probability of:

$$\frac{{e}^{p}}{1 + {e}^{p}}$$

These residuals are then determined as the difference between the values of the output values from the training data set and the probabilities. As before, a new tree can be calculated based on these residuals. This process continues until a pre-determined number of trees have been constructed or adding new trees fails to reduce the size of the residuals by more than a pre-determined margin.

The trained gradient boosting model is then applied in a similar fashion to a new observation. The process adds the initial prediction and the transformed output values of each leaf associated with the new observation to find the predicted natural logarithm of the odds. Then, the logistic function is applied to this prediction to provide a probability. If the probability is greater than 0.5, the ultimate prediction for this new observation is “true”, otherwise it is “false”.

Note that the description above provides just a general overview of a few ways to carry out gradient boosting. Other techniques are possible. Further, other types of machine learning models, such as artificial neural networks or expert systems, can be used instead or in conjunction with gradient boosting techniques. Nonetheless, gradient boosting generally works well with certain types of data and produces models that are explainable-one can analyze the model to understand how and why it is producing its answers, which might not be the case for artificial neural networks. Moreover, gradient boosting models also tend to perform well when the training data set is sparse (e.g., many observations with at least one missing input variable), whereas artificial neural networks tend to be less efficient with sparse data.

There are two popular gradient boosting frameworks that can be used to train and deploy gradient boosting models, XGBoost and LightGBM. Each is briefly discussed below for purposes of example. Nonetheless, other frameworks can be used.

### A. XGBoost

When the output variable takes on a continuous value, XGBoost builds trees by placing all residuals in a root node, and then calculates a similarity score for the residuals. The similarity score can be calculated as the square of the sum of the residuals divided by the number of residuals. In some cases, a regularization constant is also added to the denominator to reduce sensitivity to outliers and overfitting of the training data set. Regardless, the higher the similarity score, the more similar the residuals.

Then various ways of dividing the residuals into groups are considered, to see if any of these divisions results in a higher overall similarity score. This can be determined by calculating a gain for a division over the original grouping of all residuals in the root node. The gain can be the similarity score of the root node subtracted from the sum of similarity scores for each node of the divisions. Then, the division that produces the largest gain of all divisions is selected as to produce branches from the root node (i.e., each group of residuals in the selected division become a child node of the root node).

Then, the same process is performed for each of the new child nodes. If a child node contains only one residual, it cannot be further divided and becomes a leaf. Also, trees may be limited to a maximum number of levels (e.g., 4, 6, or 8) and a node at this maximum depth would also not be further divided. In some cases, XGBoost may require that a minimum number of residuals (e.g., 2, 3, 4 . . . ) be represented in each node.

Once an XGBoost tree is constructed in this fashion, some branches that produce less than a threshold gain may be pruned. The pruning process recombines them into their respective parent nodes.

Predictions are made by traversing the tree with the values of the input variable until a leaf is reached. The output value of the leaf is the sum of the residuals in that leaf divided by the number of residuals in that leaf. Again, the regularization constant may also be added to the denominator.

Like standard gradient boosting, this tree is then used to make predictions that are scaled by a learning rate. The residuals from these predictions are then used to construct the next tree, and so on. Tree construction ends when a pre-determined maximum number of trees have been constructed or the residuals become smaller than a pre-determined threshold.

When the output variable takes on a one of a discrete number of values (e.g., for classification), XGBoost maps these into a numeric range. For example, each value for a Boolean output variable would be mapped to 1.0 or 0.0. Then, the divisions are made as described above.

However, a different similar score calculation per node is used, this one being the square of the sum of residuals in the node divided by the sum over all observations the product of (i) the previous probability and (ii) the previous probability subtracted from one. The regularization constant may also be added to the denominator. Tree construction also occurs as described above, though using this different similarity score calculation to determine gain.

As noted, XGBoost may require that a minimum number of residuals be represented in each leaf. In this version of XGBoost, however, a value called “cover” is used instead of a count of residuals. Cover is the denominator of the similarity score minus the regularization constant. Leaves with less than a threshold value of cover may be removed from the tree, effectively pruning the tree. The other pruning techniques described above may also be used.

For prediction, the output value of a leaf is the sum of residuals in the leaf divided by the sum over all observations the product of (i) the previous probability and (ii) the previous probability subtracted from one. The regularization constant may also be added to the denominator. Across multiple trees, the prediction for an observation is the natural logarithm of the odds for the initial output value, added to the output values for each tree scaled by the learning rate.

The logistic function can be applied to this result in order to convert it back into a probability. Based on the value of this probability (e.g., above or below 0.5 for a binary output variable), a value of the output variable can be selected.

XGBoost also employs a number of techniques that speed up its processing for large training data sets. These techniques include using an approximate greedy algorithm for selecting divisions, weighted sketch algorithms for focusing on observations that are hard to predict, distributed training across multiple processors or computers, and/or keeping commonly-used variables and constants in the processor cache. Other techniques can also be applied.

### B. LightGBM

LightGBM also employs gradient boosting but does so in a way that generally increases training speed, reduces memory utilization, and provides improved accuracy. Particularly, rather than consider all values of an input variable, LightGBM bins these values to form a histogram, and operates on the bins rather than the values. Also, LightGBM uses exclusive feature bundling to reduce dimensions of the feature space when two or more features tend to take on mutually exclusive values. Further, LightGBM uses gradient-based one side sampling to identify the observations with the largest residuals and operate only on the observations and well as a random sampling of observations with lower residuals.

As a result, LightGBM focuses computation where it is needed most-on input variables that are dissimilar from one another and observations for which early trees have the most error. Thus, in practice, LightGBM can perform about ten times faster than other gradient boosting implementations with similar accuracy.

### III. Predicting Albuminuria

As noted, albuminuria is a condition in which albumin is detected in a patient's urine, and is indicative of chronic kidney disease (CKD). Since urine tests are not commonly ordered for most patients, CKD can progress undiagnosed and ultimately lead to kidney failure. A urine albumin-to-creatinine ratio (UACR) test with result in the range of 30-300 mg/g is referred to as microalbuminuria. A result greater than 300 mg/g is referred to as macroalbuminuria. Albuminuria is also indicative of other health conditions, including, but not limited to cardiovascular disease, hypertension, systemic vasculitis, and/or diabetes. Non-limiting examples of cardiovascular disease include, but are not limited to, heart failure such as heart failure with preserved ejection fraction (HFpEF), heart failure with mid-range ejection fraction (HFmrEF), and heart failure with reduced ejection fraction, and major adverse cardiac events (MACE), including myocardial infarction, stroke, and cardiovascular death. Further, glomerular diseases may be related as well. Generally speaking, proteinuria or albuminuria is associated with an increased risk of progression to end-stage renal disease (ESRD) and all-cause mortality.

In addition to UACR tests, estimated glomerular filtration rate (EGFR) as indicated by serum creatinine blood tests is also a biomarker for CKD (i.e., EGFR can be derived from serum/plasma creatinine). Thus, EGFR is commonly tested on a regular basis for most patients, usually in a basic metabolic panel. EGFR calculations are also based on the patient's age, gender, height, weight, and ethnicity, though other factors may be involved. EGFR may be measured in milliliters of cleansed blood per minute per body surface (mL/min/1.73 m2), with higher measurements generally indicating healthier kidney function.

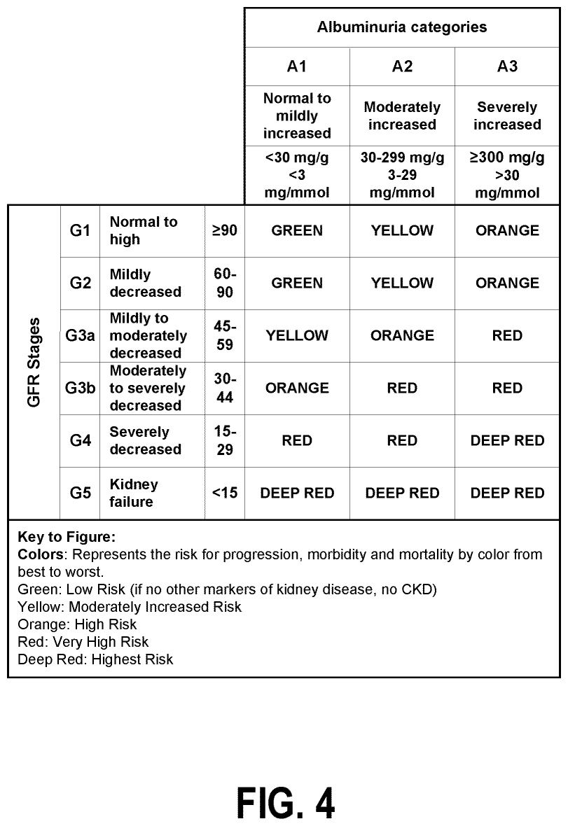

When a patient exhibits low EGFR for a period of time, a urine test to determine UACR is typically ordered. But the interaction between EGFR and UACR is complex and typically only 30%-40% of patients screened for albuminuria actually exhibit the condition. For example, as shown in FIG. 4, patients with EGFR in the range of 60-90 mL/min/1.73 m2 are at low risk for progression to CKD (defined as a EGFR of less than 60 mL/min/2.73 m2) if their UACR is less than 30 mg/g but high risk if their UACR is greater than or equal to 300 mg/g. As a consequence, considering EGFR in isolation does not accurately predict the risk for CKD.

Identifying patients with undiagnosed albuminuria based on readily available information (e.g., patient demographics, vital signs, blood tests, and/or other medical information), and for whom recent or any UACR results are not available, has a number of benefits. First, screening for albuminuria through urine tests can be made more effectively by selecting patients who are likely to exhibit the condition. Further, these patients, if ultimately diagnosed with albuminuria, can obtain treatment at an earlier stage, and before CKD progresses to kidney failure. Additionally, diagnosed patients are natural candidates for inclusion in clinical trials of new treatments and/or pharmaceuticals.

For example, a clinical trial could be designed around patients that are likely to exhibit either (i) EFGR between 20 and 30 mL/min/1.73 m2 and UACR between 30 and 5000 mg/g, or (ii) EFGR of at least 30 mL/min/1.73 m2 and UACR between 200 and 5000 mg/g. But other criteria for inclusion may be used, including different UACR and EFGR ranges.

Thus, the embodiments herein may involve developing a machine learning model that can identify patients who are likely to have undiagnosed albuminuria from a corpus of electronic health records or another data source. This model can then be validated based on clinical data, for example. Once validated, the model can be used to identify patients in hospitals and other settings who are at high risk for CKD, increased rate of EFGR decline, cardiovascular events, and renal events. These patients may be recommended for further testing, treatment, and/or inclusion in clinical trials.

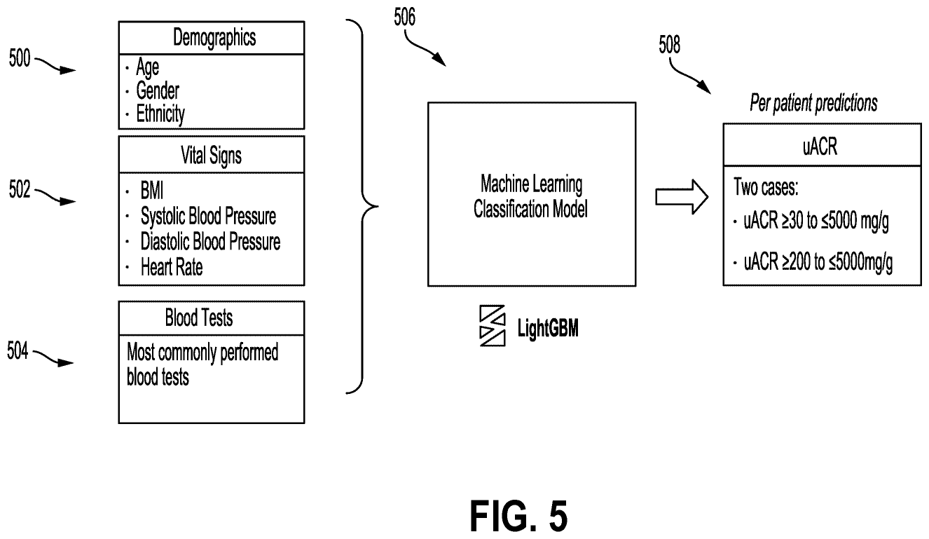

FIG. 5 depicts how the model makes predictions. Demographic data 500 (e.g., age, gender, ethnicity), vital sign data 502 (e.g., body mass index, blood pressure, heart rate), and blood test data 504 (see below) are provided as input to machine learning model 506. Other data, such as comorbidities and medications taken, may be included in the input.

Machine learning model 506 may be a classification model as shown (e.g., classifying patients into categories such as those likely to have undiagnosed albuminuria and those likely not to have the condition) or a regression model (e.g., predicting a specific UACR value per patient). Also, machine learning model 506 may be based on LightGBM as shown, or it may be based on XGBoost or some other gradient boosting technique. In alternative embodiments, machine learning model 506 may be based at least in part on an artificial neural network, expert system, or some ensemble combination of any of these models. Accordingly, machine learning model 506 may produce predictions 508, which may be a classification or a value determined by way of regression.

Data for training and validating machine learning model 506 may come from a variety of sources, including hospitals, health care providers, clinical sources, and/or insurance claims. For example, machine learning model 506 could be trained on insurance claim data, as that data may include demographics, vital signs, and blood test results for patients with and without albuminuria. This training data may be pre-processed in various ways, e.g., to remove outliers, de-skew, and/or normalize.

Machine learning model 506 as trained can then be validated on clinical data to determine to what extent it accurately predicts albuminuria in patients. Once validated, machine learning model 506 can be applied to identify patients in hospitals, using hospital services, or in other clinical or primary care settings that are candidates for further testing, treatment, or inclusion in clinical trials.

In some cases, the training data may be gathered from multiple geographic regions. However, the data may be segmented per region to develop region-specific models. In some situations, information from identified patients may be checked for novelty—e.g., whether the input variables for these patients are consistent with those in the training data. To do so, a similarity model might be applied to the information from identified patients as well as the training data, with dissimilar patients being identified for further processing before a prediction is finalized. This can be helpful if the machine learning model is trained on data from one population of individuals (e.g., located in North America) but applied to another population of individuals (e.g., located in Europe).

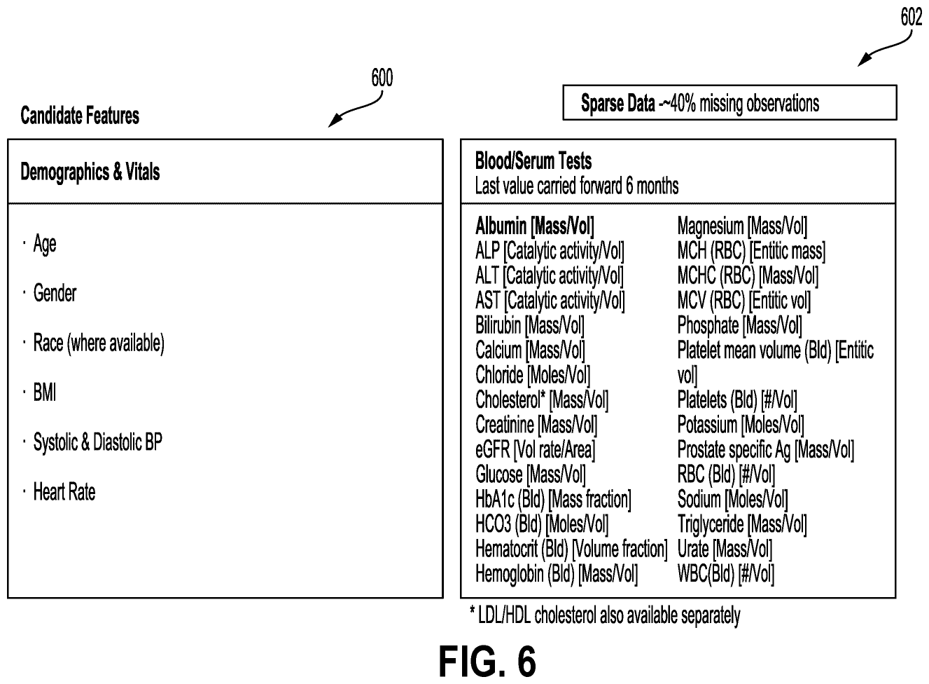

Example candidate features are shown in FIG. 6. Demographics and vitals 600 include age, gender, race, body mass index (BMI), blood pressure, and heart rate. Blood tests 602 include a number of possible measurements, including but not limited to albumin, calcium, cholesterol, EGFR, glucose, hemoglobin, magnesium, sodium, triglycerides, white blood cell (WBC) count, and so on. Other demographics, vital signs, and blood tests may be used. The output variables for this training data set, not shown, would be UACR test results (e.g., either a numeric UACR value or an indication of whether the patient from whom the observation was derived has been diagnoses with albuminuria). For example, to be included in the training data set, each observation may require a UACR test that was recorded in the last five years.

Also, some of this data may be sparse in that between 20% and 50% of input variable values may be missing across the data set. As noted, gradient boosting models typically perform well on sparse data sets.

Notably, the same or similar techniques as those described herein can also be used to predict proteinuria (UPR). Proteinuria is increased levels of protein in the urine, and can also be an indicator of CKD. A normal urine protein value in healthy adults is less than 150 mg/24 hours. Mild proteinuria is typically in the range of 150 mg/24 hours-2000 mg/24 hours, with severe proteinuria typically in the range of 2000 mg/24 hours-4000 mg/24 hours. Like albuminuria, these cutoffs can vary. While UACR is usually better at predicting CKD than UPR, UPR is still useful and a more commonly-ordered test in some regions.

Moreover, UPR values can be transformed into estimated UACR values through established equations (see Weaver, et al., Estimating Urine Albumin-to-Creatinine Ratio from Protein-to-Creatinine Ratio: Development of Equations using Same-Day Measurements, J Am Soc Nephrol. 2020 March; 31 (3): 591-601). Therefore, the UACR values used herein may be derived from UPR values, or UPR values may be used in place of the UACR values.

### IV. Example Training Data

For purposes of example, two sources of training data are considered. The first is the Limited Claims and Electronic Health Record (LCED) data set, and the second is the Optum Clinformatics Data Mart (Optum) data set. Both are from US-based patients and consist of at least hundreds of thousands of observations. Nonetheless, different training data sets may be used.

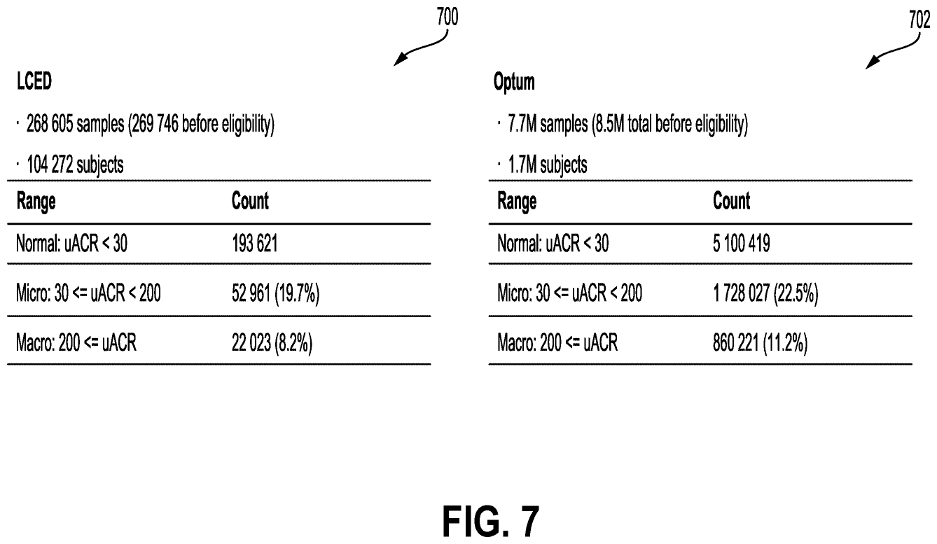

FIG. 7 presents overviews of these data sets. Overview 700 of the LCED data set indicates that it includes 268,605 observations from 104,272 patients. Approximately 19.7% of these observations are indicative of microalbuminuria, 8.2% are indicative of macroalbuminuria, and the remainder are normal. Likewise, overview 702 of the Optum data set indicates that it includes 7.7 million observations from 1.7 million patients. Approximately 22.5% of these observations are indicative of microalbuminuria, 11.2% are indicative of macroalbuminuria, and the remainder are normal.

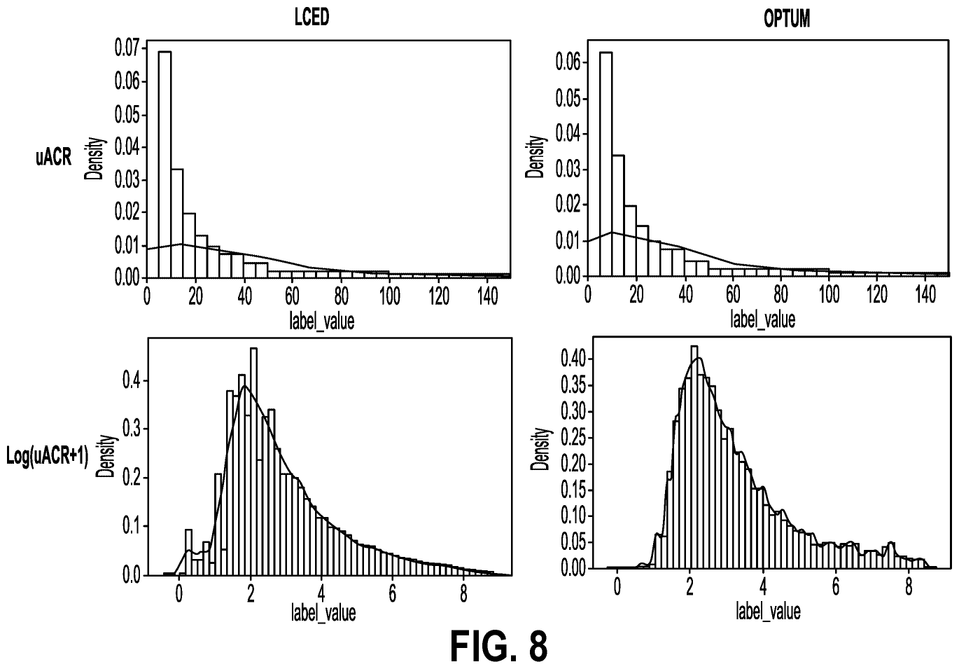

FIG. 8 depicts the distribution of UACR test results for the LCED and Optum cohorts of patients. As expected, most patients exhibit a low UACR, in the normal range. Since the distributions are heavy-tailed, FIG. 8 also provides a log transform of each to provide a better sense of the whole scope of the distributions. Since the log transforms of these data sets are less skewed, the machine learning model may be trained on the log transforms rather than the raw data. Accordingly, the machine learning model may predict log transforms of UACR values, which can then be reverted to UACR values through exponentiation.

As noted, the machine learning models of the embodiments herein may be based on regression (predicting a particular UACR value) or classification (predicting whether microalbuminuria, macroalbuminuria, or either are present). Notably, a regression-based model can easily be used for classification by mapping predicted UACR values to “true” or “false” based on the side of a cutoff value (e.g., 30 mg/g) in which they fall.

### V. Example Model Results

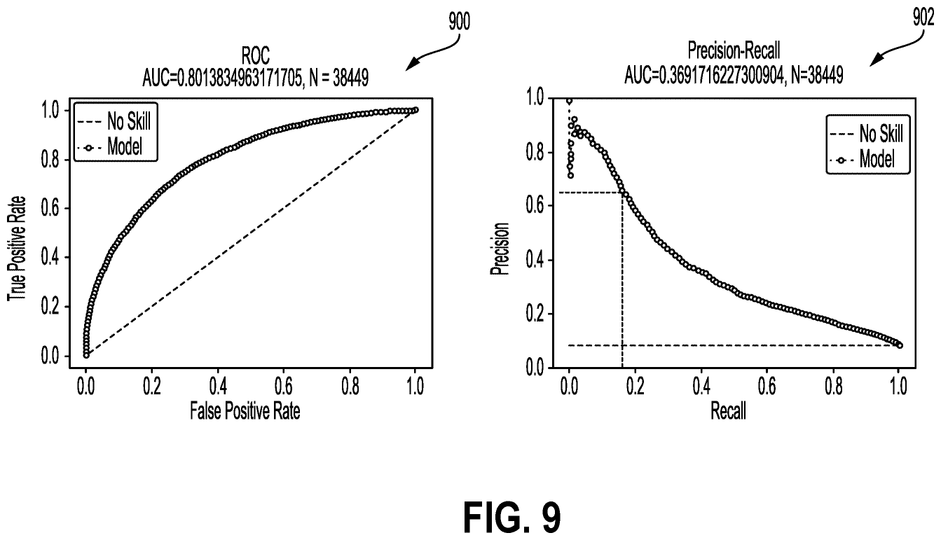

FIG. 9 depicts a receiver operating characteristic (ROC) curve 900 for the trained model, assuming that macroalbuminuria classification was used (where a UACR of at least 300 indicates macroalbuminuria). ROC curve 900 plots the true positive rate (number of true positives divided by the sum of true positives and false negatives) versus the false positive rate (number of false positives divided by the sum false positives and true negatives) of the model.

In diagnostic design, if the goal is to identify every patient who has a condition, the number of false negatives should be low and thus the true positive rate should be high. But if the goal is to identify only patients who have the condition, the number of false positives should be low, and thus the false positive rate should also be low. Setting various model parameters (including the cutoff value of UACR that divides “true” and “false” classifications) can influence both the true positive rate and the false positive rate. Note that the dashed line represents a model that performs no better than a random guess.

An ROC curve visually describes tradeoffs between the true positive rate and the false positive rate. One measure of model quality is the area under the curve (AUC) for the ROC curve. This value is typically between 0.5 and 1.0, with higher values being indicative of better model performance across various parameter settings. For instance, as shown in ROC curve 900, the AUC is about 0.801. This is indicative of a model that performs reasonably well.

FIG. 9 also depicts a precision-recall curve 902 for the model, again assuming that macroalbuminuria classification was used. Precision-recall curve 902 plots the precision (the number of true positives divided by the sum of the true positives and the false positives) versus the recall (the number of true positives divided by the sum of true positives and false negatives) of the model. Setting various model parameters (including the cutoff value of UACR that divides “true” and “false” classifications) can influence both the precision and the recall. Note that the dashed line represents a model that performs no better than a random guess.

A precision-recall curve visually describes tradeoffs between precision and recall, and is particularly useful for data sets where there are significantly more observations in one class than the other. Again, AUC can be used to evaluate model quality, with higher values indicating more quality. For instance, as shown in precision-recall curve 902, the AUC is about 0.369. This is indicative a model that performs notable better than a random guess (which has an AUC of about 0.08).

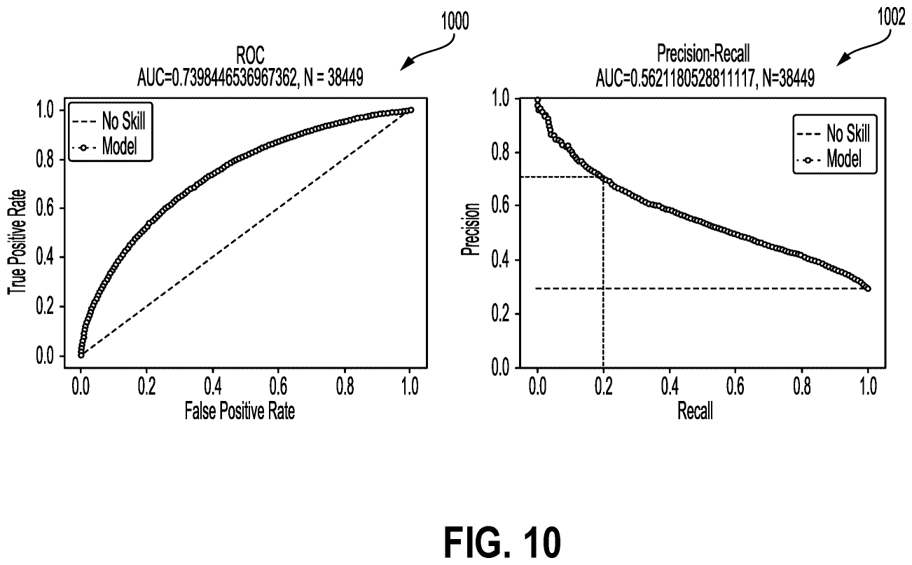

Similar to FIG. 9, FIG. 10 depicts an ROC curve 1000 for the model, assuming that microalbuminuria classification was used (where a UACR of at least 30 indicates microalbuminuria). FIG. 10 also depicts a precision-recall curve 1002 for the model, again assuming that macroalbuminuria classification was used. Again, the AUC of these curves (about 0.740 and 0.562) indicate reasonably good model performance compared to random guesses.

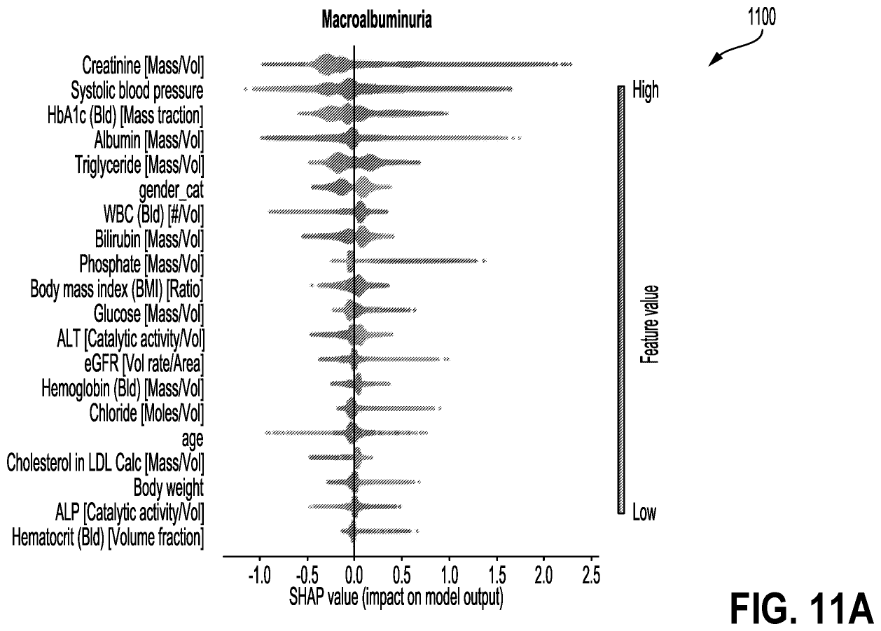

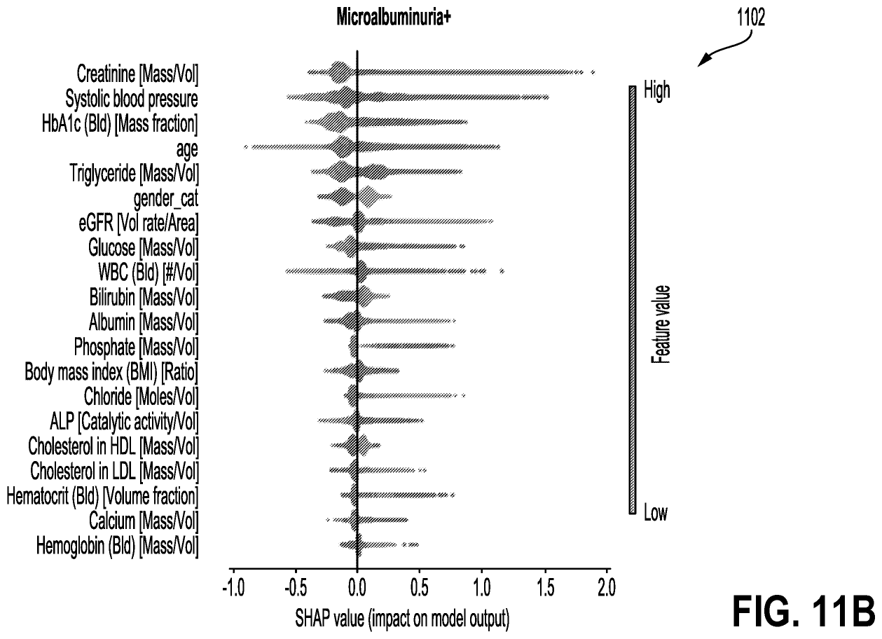

To facilitate further understanding of the trained models, FIGS. 11A and 11B provide Shapley values 1100 and 1102 of the trained model for macroalbuminuria and microalbuminuria, respectively. In short, given a particular output of a model (e.g., predicted UACR) for a particular set of input variable values, as well as the average of this output across all input values, Shapley data assigns a contribution to each of the input variables. This contribution quantifies how much each input variable contributed to the difference between the predicted output value and the average output value. Shapley data also capture possible inter-dependencies between input features such that the Shapley data is independent of the order in which the input variables are applied (should the model be sensitive to such orderings).

With respect to the UACR prediction models described herein, the Shapley values for each input variable can be used to quantify the impact of that input variable on the difference between a particular observation's predicted UACR and the average UACR of all observations. Some input variables may correlate with the particular observation's predicted UACR being higher, and others may correlate with the particular observation's predicted UACR being lower.

In Shapley values 1100 and 1102, scatterplots are shown along a corresponding x-axis for each input variable. Each value in the scatterplots corresponds to one of the observations considered by the model. The x-axis for each input variable represents the difference between predicted UACR and average UACR, and is centered on 0. The input variables at the top of the list of are generally more impactful on (e.g., more highly positively and/or negatively correlated with) predicted UACR than input variables lower in the list. In some embodiments, the input variables may be sorted top to bottom in decreasing magnitude of this impact.

For example, the three most impactful input variables in both Shapley values 1100 (for macroalbuminuria) and Shapley values 1102 (for microalbuminuria) are creatinine, systolic blood pressure, and HbA1c (glycated hemoglobin), which is shown to influence predicted UACR more than any other input variables. On the other hand, age is highly influential in predicting microalbuminuria (the fourth most influential input variable) while significantly less influential in predicting microalbuminuria (the sixteenth most influential input variable). Similar scatterplots for other input variable show how they positively or negatively impact UACR and by approximately how much. These results also indicate that EGFR alone is not a reliable predictor of albuminuria, which further justifies this more sophisticated modeling approach.

An advantage of representing Shapley data in this fashion is that a quick glance can result in a detailed understanding of why the model has predicted a specific UACR for a specific observation. For instance, it may be initially counterintuitive that age is significantly more predictive of microalbuminuria than macroalbuminuria, and such an outcome can be further investigated. Nonetheless, other types of graphs and visualizations of Shapley data may be possible.

In some cases, select Shapley values and other data could be provided to a health care professional or clinician in addition to an albuminuria prediction. These values may help explain what input variables were most influential on the prediction. For example, in initial use, the models described herein have determined that the top ten most influential features are (in descending order): creatinine (in blood/serum), systolic blood pressure, hemoglobin A1C (HbA1C), BMI, EGFR, albumin (in blood/serum), triglycerides, glucose, age, and bilirubin. These features were identified by way of a combination of Shapley analysis, frequency in datasets, and important for splits/gains when constructing decision tree.

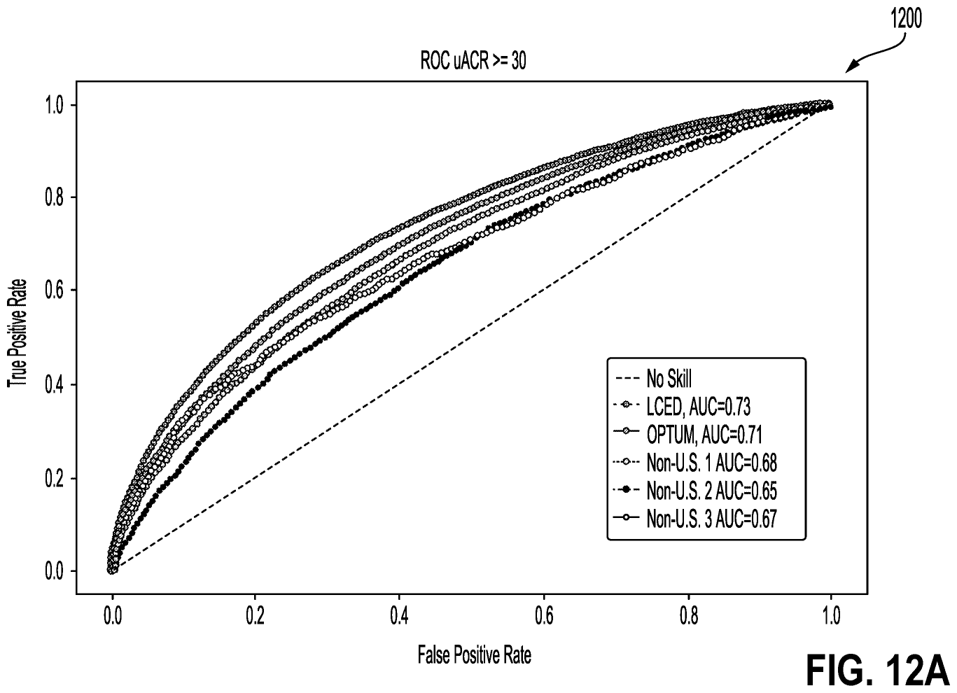

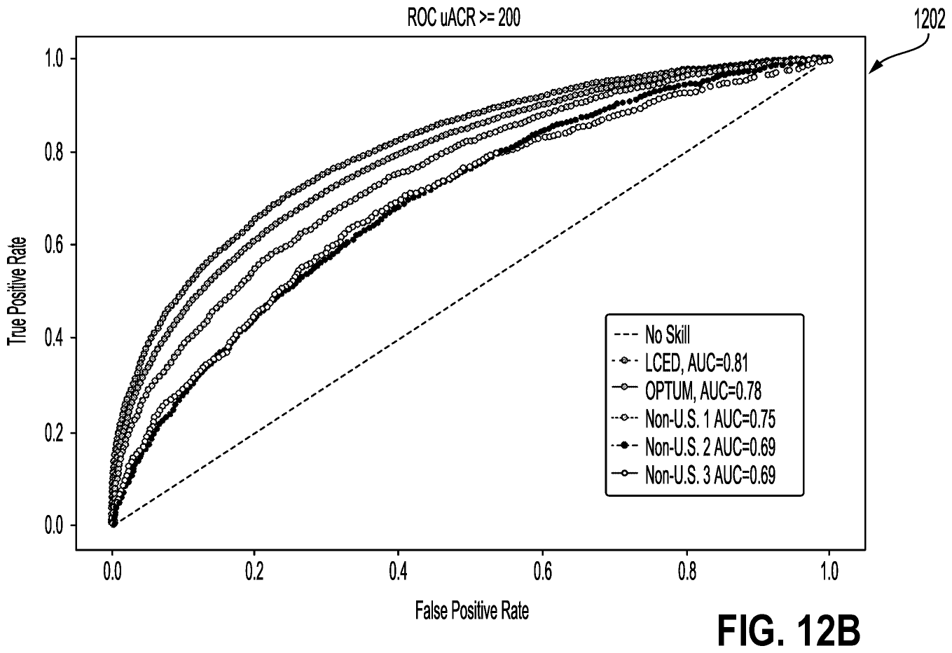

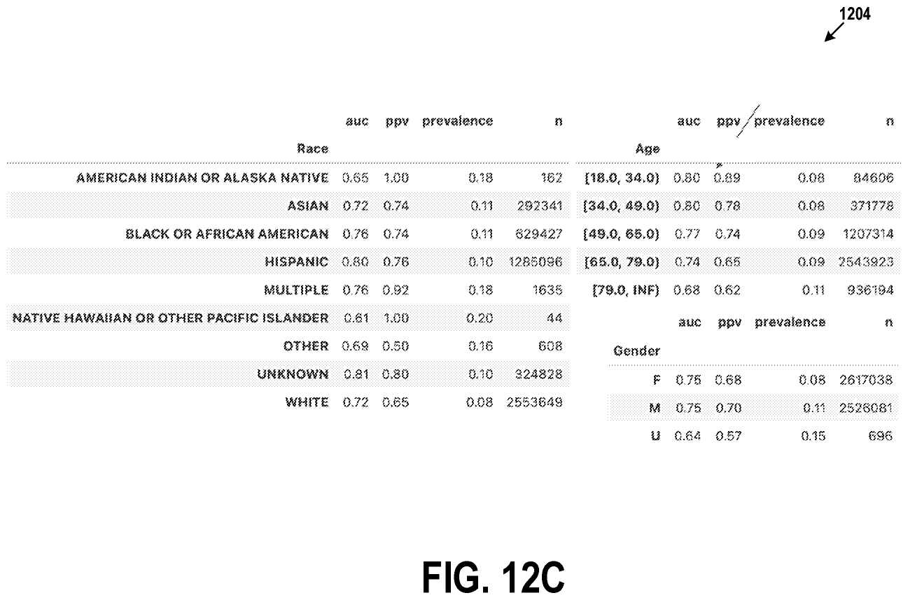

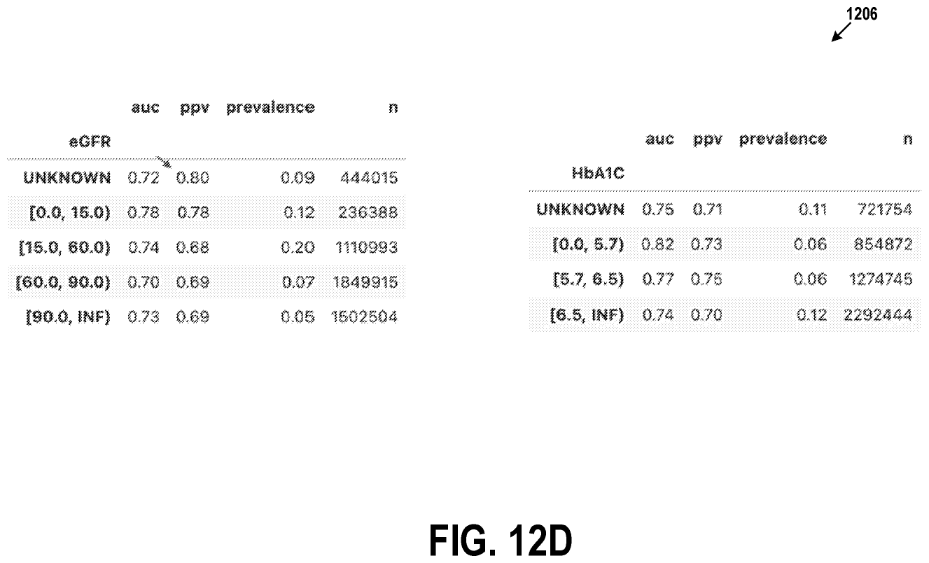

FIGS. 12A and 12B provide ROC graphs 1200 and 1202 for UACR of at least 30 and UACR of at least 200, which can be used respectively as indicators of microalbuminuria and macroalbuminuria. Each of graphs 1202 and 1204 plot data for respective models that were trained with training data sets that originated in the U.S., and used to predict albuminuria for U.S. data sets (LCED and Optum) and unnamed non-U.S. data sets (non-U.S. 1, non-U.S. 2, and non-U.S. 3). As shown in FIG. 12A, training on U.S. data results in a model that works well for predicting microalbuminuria both the U.S. data sets (AUC of 0.71-0.73) and non-U.S. data sets (AUC of 0.65-0.68). As shown in FIG. 12B, training on U.S. data results in a model that works well for predicting macroalbuminuria both the U.S. data sets (AUC of 0.78-0.81) and non-U.S. data sets (AUC of 0.69-0.75).

FIG. 12C depicts tables 1204 providing, among other factors, AUC values per race, age cohort, and gender. For the latter, M indicates male, F indicates female, and U indicates unknown. All of these AUC values indicate that the trained models perform well across races, age cohorts, and genders, though there is some variability in terms of AUC within each. FIG. 12C also evaluates the models in terms of positive prediction value (PPV), which is another term for precision (the number of true positives divided by the sum of true positives and false positives). As shown, the models also perform well in terms of PPV.

FIG. 12D depicts tables 1206 for providing AUC and PPV factors given different ranges of EGFR and HbA1C. As indicated, the models perform quite well, notably even when EGFR is unknown. This suggests that the models have significant clinical utility for albuminuria prediction in populations for which EFGR measurements are not available. Further, the models perform at about the same level of precision regardless of HbA1C levels.

### VI. Deployment Scenarios

The embodiments herein may be deployed in a number of arrangements. With regard to the training of one or more machine learning models, this may take place on one or more computing devices within a server cluster, such as in a public cloud network (e.g., Amazon AWS or Microsoft Azure) or on a private system. With regard to the execution of these models on new observations, the models could be hosted in various locations and environments.

In one possible example, the trained models may be hosted on a public cloud network or on a private network, and provide results to client devices either via a web or application interface. For instance, the client device may transmit a request to a remotely hosted model, the request containing a set of new input variables comprising the new observation. The model may take these as input and produce a corresponding output that is then transmitted to the client device in response to the request. These trained models may be operated by various entities, such as a hospital, hospital network, physician, physician network, university, pharmaceutical company, or some consortia of one or more of these to other entities.

Alternatively, the trained model may be packaged with a client application that can be downloaded and installed on a desktop, laptop, or mobile computing device. Thus, the client application would contain a user interface that allows a user to enter or otherwise indicate the input variables for a new observation. The client application would then apply the model this new observation and produce a corresponding output that is displayed and/or stored by the client device. This scenario has the advantage that a live network connection is not required to use the model.

In other alternatives, the trained model may be used to develop simple clinical prediction rules, such as a decision tree, that a health care provider can follow to predict whether a patient is likely to have albuminuria.

Other deployment scenarios may exist. Thus, the embodiments herein are not limited to these scenarios.

### VII. Example Operations

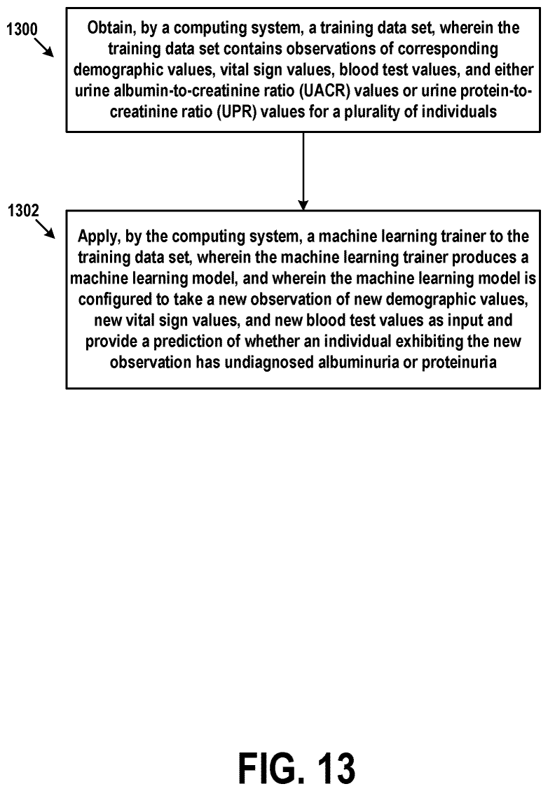

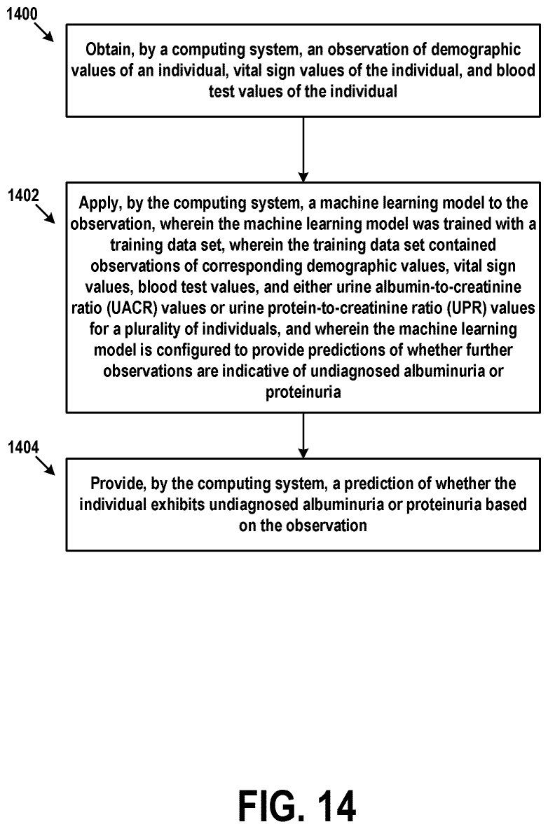

FIGS. 13 and 14 are flow charts illustrating example embodiments. The operations illustrated by FIGS. 13 and 14 may be carried out by a computing system or computing device that includes a software application configured to perform any of the embodiments herein. Non-limiting examples of the computing system or computing device include computing device 100 or server cluster 200, for example. However, the operations can be carried out by other types of devices or device subsystems. For example, the operations could be carried out by a portable computer, such as a laptop or a tablet device.

The embodiments of FIGS. 13 and 14 may be simplified by the removal of any one or more of the features shown therein. Further, these embodiments may be combined with features, aspects, and/or implementations of any of the previous figures or otherwise described herein. Such embodiments may include instructions executable by one or more processors of the one or more computing devices of the system or virtual machine or container. For example, the instructions may take the form of software and/or hardware and/or firmware instructions. In an example embodiment, the instructions may be stored on a non-transitory computer readable medium. When executed by one or more processors of the one or more computing devices, the instructions may cause the one or more computing devices to carry out various operations of the embodiments.

Block 1300 of FIG. 13 involves obtaining, by a computing system, a training data set, wherein the training data set contains observations of corresponding demographic values, vital sign values, blood test values, and either UACR or UPR values for a plurality of individuals.

Block 1302 of FIG. 13 also involves applying, by the computing system, a machine learning trainer to the training data set, wherein the machine learning trainer produces a machine learning model, and wherein the machine learning model is configured to take a new observation of new demographic values, new vital sign values, and new blood test values as input and provide a prediction of whether an individual exhibiting the new observation has undiagnosed albuminuria or proteinuria.

In some embodiments, the demographic values include ages, genders, or ethnicities of the plurality of individuals.

In some embodiments, the vital sign values include body mass indices, blood pressure readings, or heart rates of the plurality of individuals.

In some embodiments, the blood test values include creatinine levels, glycated hemoglobin levels, triglycerides, blood albumin levels, or a white blood cell count of the plurality of individuals.

In some embodiments, values within the training data set are 20%-50% populated.

In some embodiments, the machine learning model is based on gradient boosting.

In some embodiments, the prediction of whether the individual exhibiting the new observations has undiagnosed albuminuria comprises predicting whether the individual has microalbuminuria.

In some embodiments, the prediction of whether the individual exhibiting the new observations has undiagnosed albuminuria comprises predicting whether the individual has macroalbuminuria.

In some embodiments, the prediction of whether the individual exhibiting the new observations has undiagnosed albuminuria comprises or proteinuria predicting a UACR value or a UPR value for the individual.

In some embodiments, the training data set includes at least 100,000 observations gathered from medical claim records or electronic health records.

In some embodiments, the training data set includes at least 1,000,000 observations gathered from medical claim records or electronic health records.

In some embodiments, between 5% and 25% of the observations have UACR values that are indicative of albuminuria or UPR values indicative of proteinuria.

In some embodiments, the UACR values were derived mathematically from UPR values.

Block 1400 of FIG. 14 involves obtaining, by a computing system, an observation of demographic values of an individual, vital sign values of the individual, and blood test values of the individual.

Block 1402 of FIG. 14 also involves applying, by the computing system, a machine learning model to the observation, wherein the machine learning model was trained with a training data set, wherein the training data set contained observations of corresponding demographic values, vital sign values, blood test values, and UACR values for a plurality of individuals, and wherein the machine learning model is configured to provide predictions of whether further observations are indicative of undiagnosed albuminuria or proteinuria.

Block 1404 of FIG. 14 also involves providing, by the computing system, a prediction of whether the individual exhibits undiagnosed albuminuria or proteinuria based on the observation.

In some embodiments, providing the prediction comprises displaying the prediction on a graphical user interface.

In some embodiments, obtaining the observation comprises receiving the observation from a client device in communication with the computing system over a network, wherein providing the prediction comprises transmitting the prediction to the client device.

In some embodiments, the demographic values include ages, genders, or ethnicities of the plurality of individuals.

In some embodiments, the vital sign values include body mass indices, blood pressure readings, or heart rates of the plurality of individuals.

In some embodiments, the blood test values include creatinine levels, glycated hemoglobin levels, triglycerides, blood albumin levels, or a white blood cell count of the plurality of individuals.

In some embodiments, values within the training data set are 20%-50% populated.

In some embodiments, the machine learning model is based on gradient boosting.

In some embodiments, the prediction of whether the individual exhibiting the observation has undiagnosed albuminuria comprises predicting whether the individual has microalbuminuria.

In some embodiments, the prediction of whether the individual exhibiting the observation has undiagnosed albuminuria comprises predicting whether the individual has macroalbuminuria.

In some embodiments, the prediction of whether the individual exhibiting the observation has undiagnosed albuminuria or proteinuria comprises predicting a UACR value or a UPR value for the individual.

In some embodiments, the training data set includes at least 100,000 observations gathered from medical claim records or electronic health records.

In some embodiments, the training data set includes at least 1,000,000 observations gathered from medical claim records or electronic health records.

In some embodiments, between 5% and 25% of the observations have UACR values that are indicative of albuminuria or UPR values indicative of proteinuria.

In some embodiments, the UACR values were derived mathematically from UPR values.

### VIII. Quantile Regression Model to Predict Albuminuria

Instead of or in addition to the embodiments above, a quantile-based regression model may be used to predict albuminuria. Unlike a classification model that predicts whether microalbuminuria or macroalbuminuria is exhibited by a patient, this regression model predicts the 25th, 50th, and/or 75th quantiles of UACR or UPR. Other quantiles, such as the 10th and 90th, may also be predicted.

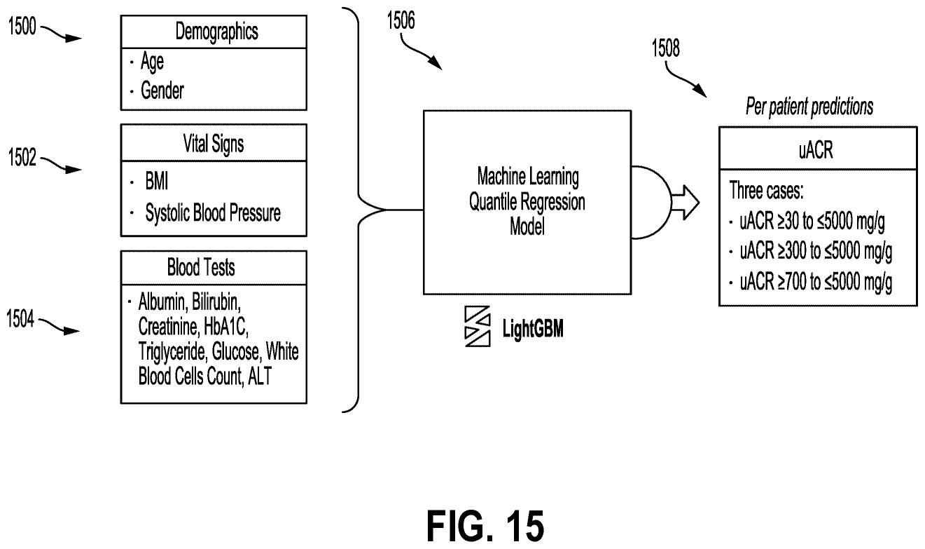

The regression-based model is depicted in FIG. 15. This model is similar to that of FIG. 5, but with some notable differences.

Demographic data 1500 (here, just age and gender), vital sign data 1502 (here, just body mass index and systolic blood pressure), and blood test data 1504 are provided as input to machine learning model 1506. Other data, such as comorbidities and medications taken, may be included in the input. Furthermore, additional data may be included, such as data discussed in the context of FIG. 5.

For this model, blood test data 1504 focuses on levels of albumin, bilirubin, creatinine, HbA1C, triglycerides, glucose, white blood cell count, and ALT. Other combinations of markers may be used.

Machine learning model 1506 may be a quantile regression model as shown. Also, machine learning model 1506 may be based on LightGBM as shown, or it may be based on XGBoost or some other gradient boosting technique. In alternative embodiments, machine learning model 1506 may be based at least in part on an artificial neural network, expert system, or some ensemble combination of any of these models. Accordingly, machine learning model 1506 may produce predictions 1508, which may be one or more quantiles of UACR or UPR.

Quantile regression is a statistical technique used to estimate the relationship between one or more input variables and an output variable across one or more quantiles of the output variable. Unlike least squares regression, which estimates the mean of the output variable as a function of the input variables, quantile regression estimates the conditional quantiles of the output variable. For example, the model can be configured to estimate the 25th, 50th, and 75th percentiles of the output variable for different values of the input variables. This allows determination of how the relationship between the input and output variables changes across different parts of the distribution of the output variable.

Such a model may operate at least in part by minimizing a loss function that penalizes the differences between the predicted and observed quantiles. For example, a quantile regression model for a quantile t could be:

$${Q}_{τ} ( {y}_{i} ) = {β}_{0} ( τ ) + {β}_{1} ( τ ) {x}_{i 1} + … + {β}_{p} ( τ ) {x}_{ip} , \qquad i = 1 , … , n$$

In this equation, the βⱼ(τ) coefficients are functions of the quantile rather than constants. Finding the values for these coefficients at a particular quantile is similar to that of linear regression, except that the median absolute deviation (MAD) is minimized. Particularly:

$$MAD = \frac{1}{n} {\sum }_{i = 1}^{n} {ρ}_{τ} ( {y}_{i} - {Q}_{τ} ( {y}_{i} ) )$$

The function ρ_{τ} is defined as:

$${ρ}_{τ} ( u ) = τ max ( u , 0 ) + ( 1 - τ ) max ( - u , 0 )$$

Thus, ρ_{τ} gives asymmetric weights to the error depending on the quantile and the overall sign of the error. This means that if the error is positive, then ρ_{τ} multiples the error by τ, and if the error is negative, then ρ_{τ} multiplies the error by (1−t). For instance, to determine the median of the 25th quantile, 75% of the errors should be positive and 25% should be negative. In order to find the smallest MAD where this property is true, weights are added to the errors. In the case of the 25th quantile, a weight of 0.75 is added the negative errors and a weight of 0.25 is added to the positive errors.

This quantile regression technique serves to describe the relationship between the input and output variables at each quantile, and can be used to make predictions about the output variable at specific quantiles for new values of the input variable. Thus, quantile regression is a useful tool for exploring how the relationship between input and output variables vary across the distribution of the output variable.

As was the case for machine learning model 506, data for training and validating machine learning model 1506 may come from a variety of sources, including hospitals, health care providers, clinical sources, and/or insurance claims. For example, machine learning model 1506 could be trained on insurance claim data, as that data may include demographics, vital signs, and blood test results for patients with and without albuminuria. This training data may be pre-processed in various ways, e.g., to remove outliers, de-skew, and/or normalize.

Machine learning model 1506 as trained can then be validated on clinical data to determine to what extent it accurately predicts quantiles of UACR or UPR in patients. Once validated, machine learning model 1506 can be applied to identify patients in hospitals, using hospital services, or in other clinical or primary care settings that are candidates for further testing, treatment, or inclusion in clinical trials.

In comparison to machine learning model 506, machine learning model 1506 predicts quantiles of UACR or UPR rather than classifying patients of having microalbuminuria, macroalbuminuria, or neither. Thus, a user may select a quantile and a threshold with which to classify patients into risk groups. For example, using the 25th quantile, there is a 75% probability that the actual value will be above the predicted value, implying a high precision. Then the predicted UACR or UPR value at the quantile is compared with the macroalbuminuria threshold of 300, to determine whether the patient is eligible for treatment or inclusion in a study. In contrast, using the 50th quantile reduces precision but increases recall, so it may be more appropriate for different purposes very high UACR or UPR values. The difference between quantile predictions also provides an estimate of how confident the model is in the predictions (e.g., a lower difference implies higher confidence).

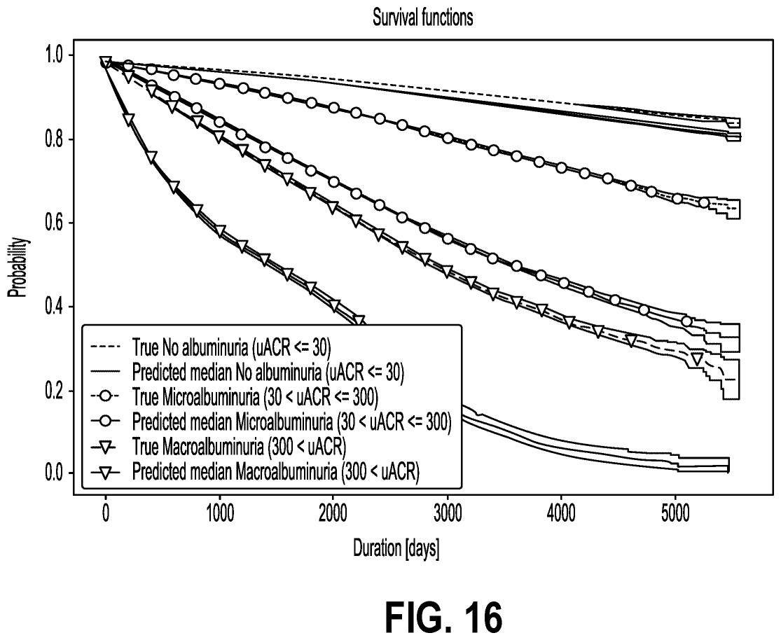

FIG. 16 provides an example of the results that can be produced by such a system. The graph in this figure plots days survived on the x-axis versus probability on the y-axis for various situations. The dashed line is for individuals truly without albuminuria (e.g., UACR≤30), and the solid line is for individuals predicted to not have albuminuria (e.g., median predicted UACR≤30). The dashed line with circles is for individuals truly with microalbuminuria (e.g., 30<UACR≤300), and the solid line with circles is for individuals predicted to have microalbuminuria (e.g., median predicted 30<UACR≤300). The dashed line with triangles is for individuals truly with macroalbuminuria (e.g., 300<UACR), and the solid line with triangles is for individuals predicted to have macroalbuminuria (e.g., median predicted 300<UACR).

As can be seen from FIG. 16, the predicted median UACR generally agrees with the survival rates for corresponding true UACR values. In these curves, the estimated survival (e.g., a Kaplan-Meier estimate) is plotted for patients that have performed a urine test (these are the “true” dashed lines). The estimated survival for the patients the model identifies as having no albuminuria, microalbuminuria, or macroalbuminuria are also plotted based on the predicted quantile. But due to the nature of quantile regression, only predictions where the model is most certain of the values (e.g., in the case of 25% or 50% quantile) are provided, so patients with uncertain values will not be included in the estimate. The model then identifies patients with the worst renal function, as it manifests more clearly in the laboratory tests, and these patients will then also have the worst survival rates. This is why the predicted survival for the patients is different from the true survival rates, because they represent just a subset of all patients of that category of albuminuria, most likely those with the worst renal function. Regardless, for the primary use case, these are the desired survival curves.

### IX. Additional Example Operations

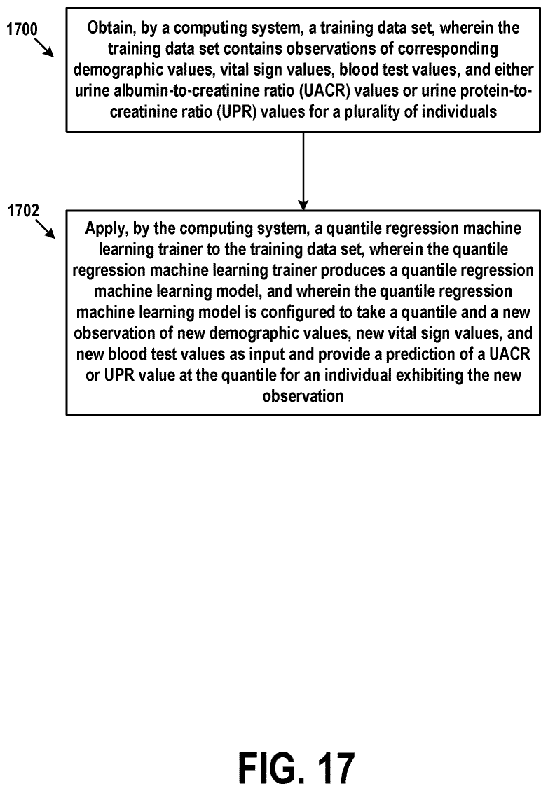

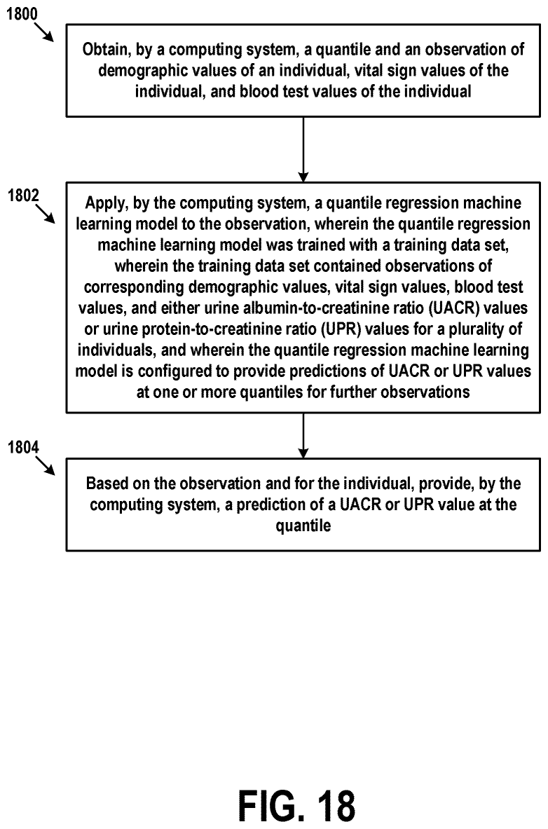

FIGS. 17 and 18 are flow charts illustrating example embodiments. The operations illustrated by FIGS. 17 and 18 may be carried out by a computing system or computing device that includes a software application configured to perform any of the embodiments herein. Non-limiting examples of the computing system or computing device include computing device 100 or server cluster 200, for example. However, the operations can be carried out by other types of devices or device subsystems. For example, the operations could be carried out by a portable computer, such as a laptop or a tablet device.

The embodiments of FIGS. 17 and 18 may be simplified by the removal of any one or more of the features shown therein. Further, these embodiments may be combined with features, aspects, and/or implementations of any of the previous figures or otherwise described herein. Notably, these embodiments can be used with any relevant features discussed in the context of FIGS. 13 and 14.

Such embodiments may include instructions executable by one or more processors of the one or more computing devices of the system or virtual machine or container. For example, the instructions may take the form of software and/or hardware and/or firmware instructions. In an example embodiment, the instructions may be stored on a non-transitory computer readable medium. When executed by one or more processors of the one or more computing devices, the instructions may cause the one or more computing devices to carry out various operations of the embodiments.

Block 1700 of FIG. 17 involves obtaining, by a computing system, a training data set, wherein the training data set contains observations of corresponding demographic values, vital sign values, blood test values, and either urine albumin-to-creatinine ratio (UACR) values or urine protein-to-creatinine ratio (UPR) values for a plurality of individuals.

Block 1702 of FIG. 17 also involves applying, by the computing system, a quantile regression machine learning trainer to the training data set, wherein the quantile regression machine learning trainer produces a quantile regression machine learning model, and wherein the quantile regression machine learning model is configured to take a quantile and a new observation of new demographic values, new vital sign values, and new blood test values as input and provide a prediction of a UACR or UPR value at the quantile for an individual exhibiting the new observation.

Block 1800 of FIG. 18 involves obtaining, by a computing system, a quantile and an observation of demographic values of an individual, vital sign values of the individual, and blood test values of the individual.

Block 1802 of FIG. 18 also involves applying, by the computing system, a quantile regression machine learning model to the observation, wherein the quantile regression machine learning model was trained with a training data set, wherein the training data set contained observations of corresponding demographic values, vital sign values, blood test values, and either urine albumin-to-creatinine ratio (UACR) values or urine protein-to-creatinine ratio (UPR) values for a plurality of individuals, and wherein the quantile regression machine learning model is configured to provide predictions of UACR or UPR values at one or more quantiles for further observations.

Block 1804 of FIG. 18 also involves, based on the observation and for the individual, providing, by the computing system, a prediction of a UACR or UPR value at the quantile.

### X. Example User Interface and Workflow

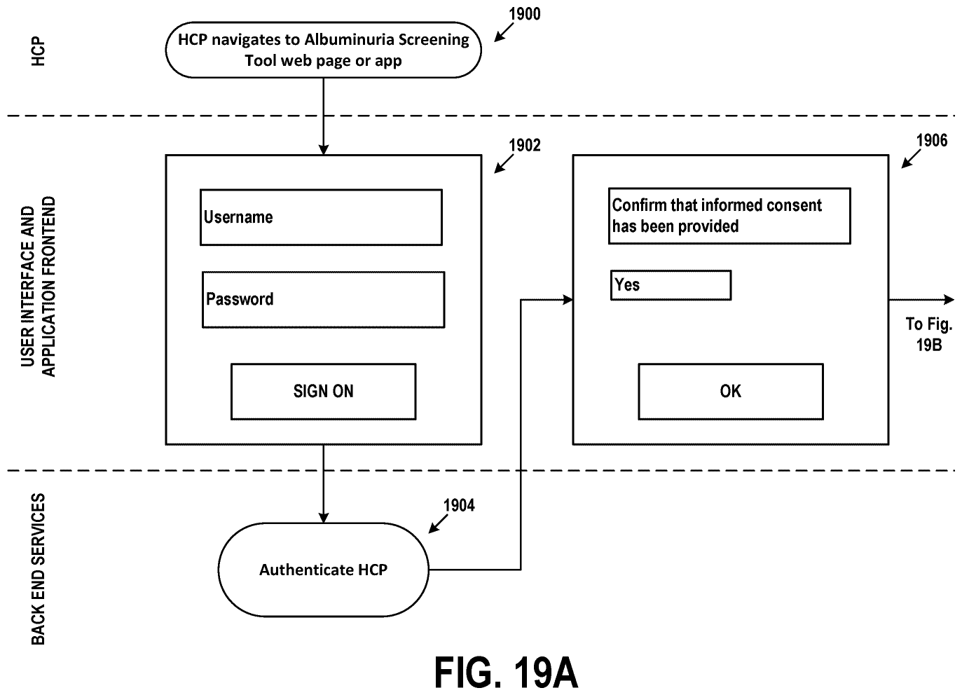

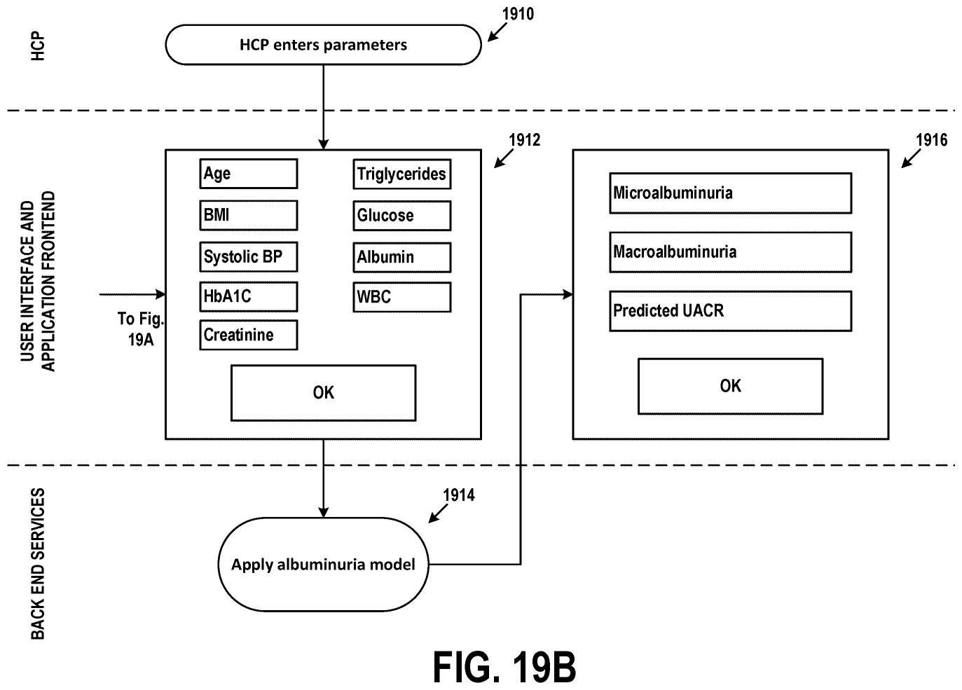

FIGS. 19A and 19B depict an example workflow for software that uses input from a health care provider (HCP) or other user to execute a model that predicts factors relating to albuminuria. This software includes a user interface and application frontend that may be web-based or a standalone application (e.g., a mobile app). The software also includes backend services (e.g., disposed upon a remote server) that perform authentication and model execution functions. Nonetheless, the model may also include other features and functions not shown in these figures.

Turning to FIG. 19A, at step 1900 the HCP navigates to the albuminuria screening tool web page or application. The HCP is presented with screen 1902 on a user interface. Screen 1902 includes text boxes in which the HCP may enter their secure credentials (e.g., userid and password).

After actuating the “SIGN ON” button of screen 1902, the HCP's credentials (or some representation thereof) are transmitted to the backend services. In response, the backend services perform step 1904 to authenticate the HCP.

Assuming that this authentication is successful, the HCP is presented with screen 1906 on the user interface. Screen 1906 requests that the HCP confirm that the patient has provided informed consent for their data to be entered. Assuming that this is the case, the HCP actuates the “OK” button and control passes to screen 1912 of FIG. 19B.

Turning to FIG. 19B, at step 1910 the HCP enters parameters from the patient's data into text boxes of screen 1912. As shown, these parameters may include the patient's age, BMI, systolic blood pressure, HbA1C, creatinine, triglycerides, glucose, albumin, and WBC values. Nonetheless, more or fewer parameters may be entered, and other parameters could be used.

After the HCP actuates the “OK” button, step 1914 is performed by the back end services. In particular, one or more of the albuminuria prediction models discussed herein are performed on the parameters. As noted, these models can be used to predict whether the patient is likely to have microalbuminuria, macroalbuminuria, or neither.

Accordingly, the output of the model is used to populate screen 1916. This screen may display, for example, whether the patient is predicted to have microalbuminuria or macroalbuminuria. The screen may also display the predicted UACR of the patient.

In addition to the features and functions described, the frontend and the backend services may also support the HCP logging out of the application, help screens, and so on.

### XI. Closing

The present disclosure is not to be limited in terms of the particular embodiments described in this application, which are intended as illustrations of various aspects. Many modifications and variations can be made without departing from its scope, as will be apparent to those skilled in the art. Functionally equivalent methods and apparatuses within the scope of the disclosure, in addition to those described herein, will be apparent to those skilled in the art from the foregoing descriptions. Such modifications and variations are intended to fall within the scope of the appended claims.

The above detailed description describes various features and operations of the disclosed systems, devices, and methods with reference to the accompanying figures. The example embodiments described herein and in the figures are not meant to be limiting. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations.

With respect to any or all of the message flow diagrams, scenarios, and flow charts in the figures and as discussed herein, each step, block, and/or communication can represent a processing of information and/or a transmission of information in accordance with example embodiments. Alternative embodiments are included within the scope of these example embodiments. In these alternative embodiments, for example, operations described as steps, blocks, transmissions, communications, requests, responses, and/or messages can be executed out of order from that shown or discussed, including substantially concurrently or in reverse order, depending on the functionality involved. Further, more or fewer blocks and/or operations can be used with any of the message flow diagrams, scenarios, and flow charts discussed herein, and these message flow diagrams, scenarios, and flow charts can be combined with one another, in part or in whole.

A step or block that represents a processing of information can correspond to circuitry that can be configured to perform the specific logical functions of a herein-described method or technique. Alternatively or additionally, a step or block that represents a processing of information can correspond to a module, a segment, or a portion of program code (including related data). The program code can include one or more instructions executable by a processor for implementing specific logical operations or actions in the method or technique. The program code and/or related data can be stored on any type of computer readable medium such as a storage device including RAM, a disk drive, a solid-state drive, or another storage medium.

The computer readable medium can also include non-transitory computer readable media such as non-transitory computer readable media that store data for short periods of time like register memory and processor cache. The non-transitory computer readable media can further include non-transitory computer readable media that store program code and/or data for longer periods of time. Thus, the non-transitory computer readable media may include secondary or persistent long-term storage, like ROM, optical or magnetic disks, solid-state drives, or compact disc read only memory (CD-ROM), for example. The non-transitory computer readable media can also be any other volatile or non-volatile storage systems. A non-transitory computer readable medium can be considered a computer readable storage medium, for example, or a tangible storage device.

Moreover, a step or block that represents one or more information transmissions can correspond to information transmissions between software and/or hardware modules in the same physical device. However, other information transmissions can be between software modules and/or hardware modules in different physical devices.

The particular arrangements shown in the figures should not be viewed as limiting. It should be understood that other embodiments could include more or less of each element shown in a given figure. Further, some of the illustrated elements can be combined or omitted. Yet further, an example embodiment can include elements that are not illustrated in the figures.

While various aspects and embodiments have been disclosed herein, other aspects and embodiments will be apparent to those skilled in the art. The various aspects and embodiments disclosed herein are for purpose of illustration and are not intended to be limiting, with the true scope being indicated by the following claims.

1-15. (canceled)

16. A method comprising:
obtaining, by a computing system, an observation of demographic values of an individual, vital sign values of the individual, and blood test values of the individual;
applying, by the computing system, a machine learning model to the observation, wherein the machine learning model was trained with a training data set, wherein the training data set contained observations of corresponding demographic values, vital sign values, blood test values, and either urine albumin-to-creatinine ratio (UACR) values or urine protein-to-creatinine ratio (UPR) values for a plurality of individuals, and wherein the machine learning model is configured to provide predictions of whether further observations are indicative of undiagnosed albuminuria or proteinuria; and
providing, by the computing system, a prediction of whether the individual exhibits undiagnosed albuminuria or proteinuria based on the observation.

17. The method of claim 16, wherein providing the prediction comprises displaying the prediction on a graphical user interface.

18. The method of claim 16, wherein obtaining the observation comprises receiving the observation from a client device in communication with the computing system over a network, and wherein providing the prediction comprises transmitting the prediction to the client device.

19. The method of claim 16, wherein the demographic values include ages, genders, or ethnicities of the plurality of individuals.

20. The method of claim 16, wherein the vital sign values include body mass indices, blood pressure readings, or heart rates of the plurality of individuals.

21. The method of claim 16, wherein the blood test values include creatinine levels, glycated hemoglobin levels, triglycerides, blood albumin levels, or a white blood cell count of the plurality of individuals.

22. The method of claim 16, wherein values within the training data set are 20%-50% populated.

23. The method of claim 16, wherein the machine learning model is based on gradient boosting.

24. The method of claim 16, wherein the prediction of whether the individual exhibiting the observation has undiagnosed albuminuria comprises predicting whether the individual has microalbuminuria.

25. The method of claim 16, wherein the prediction of whether the individual exhibiting the observation has undiagnosed albuminuria comprises predicting whether the individual has macroalbuminuria.

26. The method of claim 16, wherein the prediction of whether the individual exhibiting the observation has undiagnosed albuminuria or proteinuria comprises predicting a UACR value or a UPR value for the individual.

27. The method of claim 16, wherein the training data set includes at least 100,000 observations gathered from medical claim records or electronic health records.

28. The method of claim 16, wherein the training data set includes at least 1,000,000 observations gathered from medical claim records or electronic health records.

29. The method of claim 16, wherein between 5% and 25% of the observations have UACR values that are indicative of albuminuria or UPR values indicative of proteinuria.

30. The method of claim 16, further comprising:
based on the prediction indicating that the individual exhibits undiagnosed albuminuria or proteinuria, recommending that the individual be treated for albuminuria or proteinuria.

31. The method of claim 16, further comprising:
based on the prediction indicating that the individual exhibits undiagnosed albuminuria or proteinuria, recommending that the individual be enrolled in a clinical trial related to albuminuria or proteinuria.

32. The method of claim 16, wherein the UACR values were derived mathematically from UPR values.

33. (canceled)

34. A method comprising:
obtaining, by a computing system, a quantile and an observation of demographic values of an individual, vital sign values of the individual, and blood test values of the individual;
applying, by the computing system, a quantile regression machine learning model to the observation, wherein the quantile regression machine learning model was trained with a training data set, wherein the training data set contained observations of corresponding demographic values, vital sign values, blood test values, and either urine albumin-to-creatinine ratio (UACR) values or urine protein-to-creatinine ratio (UPR) values for a plurality of individuals, and wherein the quantile regression machine learning model is configured to provide predictions of UACR or UPR values at one or more quantiles for further observations; and
based on the observation and for the individual, providing, by the computing system, a prediction of a UACR or UPR value at the quantile.

35. A non-transitory computer-readable medium, having stored thereon program instructions that, upon execution by a computing system, cause the computing system to perform operations of any of claims 16-34 comprising:
obtaining an observation of demographic values of an individual, vital sign values of the individual, and blood test values of the individual;
applying a machine learning model to the observation, wherein the machine learning model was trained with a training data set, wherein the training data set contained observations of corresponding demographic values, vital sign values, blood test values, and either urine albumin-to-creatinine ratio (UACR) values or urine protein-to-creatinine ratio (UPR) values for a plurality of individuals, and wherein the machine learning model is configured to provide predictions of whether further observations are indicative of undiagnosed albuminuria or proteinuria; and
providing a prediction of whether the individual exhibits undiagnosed albuminuria or proteinuria based on the observation.

36. (canceled)

37. The non-transitory computer-readable medium of claim 35, wherein between 5% and 25% of the observations have UACR values that are indicative of albuminuria or UPR values indicative of proteinuria.
